(19) United States
(12) Patent Application Publication
(10) Pub. No.: US 2023/0414762 A1
(43) Pub. Date: Dec. 28, 2023

(54) PROSTATE-SPECIFIC MEMBRANE ANTIGEN (PSMA)-TARGETED PRODRUG FOR SELECTIVE KILLING OF CELLS EXPRESSING PSMA

(71) Applicant: The Johns Hopkins University, Baltimore, MD (US)

(72) Inventors: Srikanth Boinapally, Baltimore, MD (US); IL Minn, Ellicott City, MD (US); Martin G. Pomper, Baltimore, MD (US)

(21) Appl. No.: 18/253,231

(22) PCT Filed: Nov. 17, 2021
(86) PCT No.: PCT/US2021/059663

§ 371 Date: May 17, 2023

Related U.S. Application Data
(60) Provisional application No. 63/114,583, filed on Nov. 17, 2020.

(51) Int. Cl. A61K 47/54; A61K 47/65; A61K 45/06; A61P 35/04

(52) U.S. Cl. CPC A61K 47/542; A61K 47/65; A61K 45/06; A61P 35/04; A61K 47/545

(57) ABSTRACT

A non-radioactive prodrug comprising a PSMA-targeted moiety, a cleavable linker, and an antineoplastic agent capable of selectively killing PSMA-expressing cells and methods of treating a disease or condition associated with PSMA-expressing tumors or cells is disclosed.

### FEDERALLY SPONSORED RESEARCH OR DEVELOPMENT

This invention was made with government support under grant CA058236 awarded by the National Institutes of Health. The government has certain rights in the invention.

### BACKGROUND

PSMA is over-expressed on the membrane of aggressive forms of prostate cancer, Foss et al., 2012; Kiess et al., 2015, many other human cancers, Nimmagadda et al., 2018, and endothelial cells of tumor neovasculature. Foss et al., 2012. In addition, PSMA can be engineered into any target cells as a reporter. Castanares et al., 2014. This property has made PSMA an excellent marker for targeted therapy.

Radiopharmaceutical therapies (RPT) targeting advanced prostate cancer have been tested in many clinical trials with unprecedented success. Rowe et al., 2016. RPTs using beta particle emitters (e.g. Lu-177) have shown great efficacy with minimal side effects, but a majority of treated patients relapse, requiring repeated treatments. Miyahira et al., 2018. Trials with alpha particle emitters (e.g. Ac-225) have shown enhanced efficacy, but also toxicity, including lethal renal failure, xerostomia, and alacrima. Kratochwil et al., 2017; Kratochwil et al., 2016; Kiess et al., 2016. These toxicities are due to the expression of PSMA in some normal tissues and the inability for current RPT agents to distinguish PSMA-expressing cancer cells from normal cells. In addition, RPT tends to be used primarily in major medical centers with the capacity to work with the high levels of radioactivity required. Accordingly, there is a need for developing new PSMA-targeted agents that are non-radioactive and that are capable of selectively targeting cancers.

### SUMMARY

In some aspects, the presently disclosed subject matter provides a compound comprising a PSMA-targeting moiety (T), a cleavable linker (L₁), and an antineoplastic agent (A) of formula (I): A-L₁-T (I). In some aspects, the compound further comprises a non-cleavable linker (L₂), wherein the compound of formula (I) has the following general structure: A-L₁-L₂-T (I). In some aspects, the compound further comprises a spacer (S), wherein the compound of formula (I) has the following general structure: A-S-L₁-T (I). In some aspects, the compound further comprises anon-cleavable linker (L₂) and a spacer (S), wherein the compound of formula (I) has the following general structure: A-S-L₁-L₂-T (I).

In some aspects, the PSMA-targeting moiety comprises a lysine (Lys)-Urea-glutamate (Glu)-based PSMA targeting moiety. In some aspects, the non-cleavable linker is derived from disuccinimidyl suberate or polyethylene glycol (PEG). In some aspects, the cleavable linker (L₁) comprises a cathepsin-cleavable linker. In certain aspects, the cathepsin-cleavable linker comprises one or more amino acids selected from the group consisting of valine (Val), citrulline (Cit), phenylalanine (Phe), lysine (Lys), glycine (Gly), alanine (Ala), asparagine (Asn), and combinations thereof. In particular aspects, the cathepsin-cleavable linker comprises an amino acid combination selected from the group consisting of Val-Cit, Phe-Lys, Val-Ala, Val-Gly, Gly-Gly, Gly-Gly-Gly, and Ala-Ala-Asn. In some aspects, the spacer (S) comprises a moiety selected from the group consisting of (para-aminobenzylcarbamate) (PABC), a precursor derived from para-nitrophenol (PNP), and combinations thereof.

In some aspects, the antineoplastic agent comprises a cytotoxic agent. In some aspects, the cytotoxic agent comprises a naturally-occurring or a synthetic tubulin inhibitor. In certain aspects, the cytotoxic agent is selected from the group consisting of monomethyl auristatin E (MMAE), monomethyl auristatin F (MMAF), monomethyl Dolastatin 10, maytansinoid, DM1, DM4, cabazitaxel, paclitaxel, and 7-ethyl-10-hydroxycamptothecin (SN-38), one or more topoisomerase inhibitors, plant derived natural phenols, one or more PARP inhibitors, one or more amatoxins, and Pseudomonas exotoxin A. In particular aspects, the synthetic tubulin inhibitor comprises monomethyl auristatin E (MMAE).

In some aspects, the compound of formula (I) comprises:

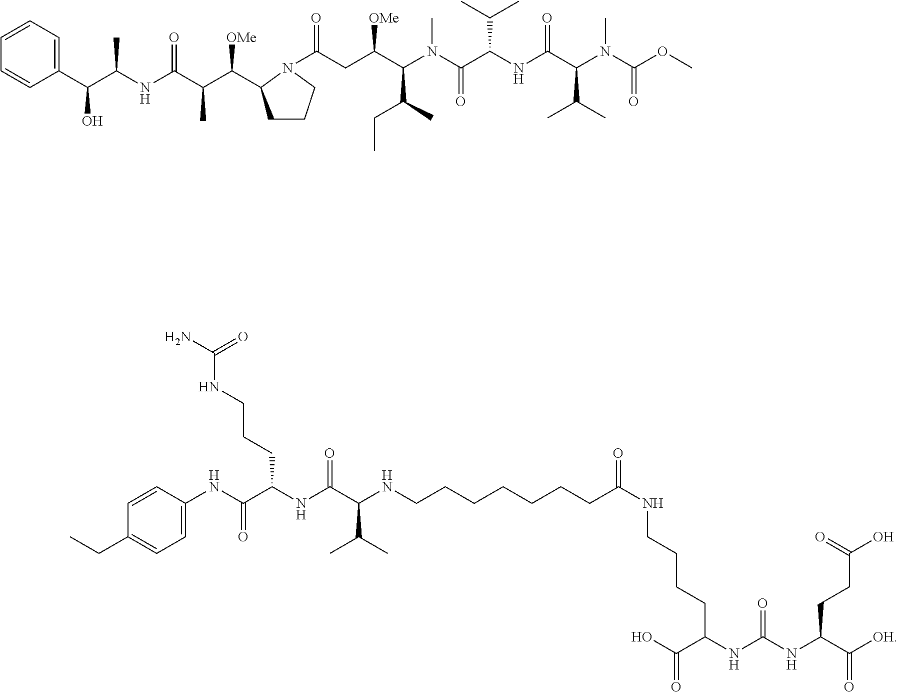

In other aspects, the presently disclosed subject matter provides a method for treating a disease or condition associated with one or more PSMA expressing tumors or cells, the method comprising administering a therapeutically effective amount of a compound of formula (I) to a subject in need of treatment thereof. In some aspects, the disease or condition comprises a cancer. In certain aspects, the cancer is selected from the group consisting of prostate cancer, renal cancer, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, adenomas, and tumor neovasculature. In particular aspects, the cancer comprises prostate cancer. In yet more particular aspects, the prostate cancer comprises metastatic castration-resistant prostate cancer. In other aspects, the cancer comprises breast cancer.

In some aspects, the method further comprises administering a compound of formula (I) in combination with one or more additional cancer treatments. In some aspects, the one or more additional cancer treatments is selected from the group consisting of chemotherapy, radiotherapy, immunotherapy, proton therapy, photodynamic therapy, and surgery.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1a and FIG. 1b demonstrate that SBPD-1 is a PSMA-targeted prodrug that releases MMAE through the action of cathepsin B. (FIG. 1a) Structures of SBPD-1 and SBPD-2. PSMA-targeting moiety (green), linker (black), cathepsin B cleavable linker (Blue), and MMAE (red). (FIG. 1b) Release of MMAE upon treatment of SBPD-1 with recombinant cathepsin B represented by decrease of intact SBPD-1 (upper). Standard curve generated for the quantification of intact SBPD-1 (lower);

FIG. 2a, FIG. 2b, and FIG. 2c demonstrate that SBPD-1 selectively kills PSMA-expressing cancer cells. Sigmoidal curves of MMAE (FIG. 2a), SBPD-1 (FIG. 2b), and SBPD-2 (FIG. 2c) for cytotoxic activity against PSMA+ PC3 PIP and PSMA− PC3 flu cell lines;

FIG. 3a and FIG. 3b demonstrate that SBPD-1 is relatively less stable than SBPD-2 in murine serum, but SBPD-1 (FIG. 3a) is stable in human serum. SBPD-1 (FIG. 3a) and SBPD-2 (FIG. 3b) were quantified by HPLC at various times after incubation with murine serum;

FIG. 4a and FIG. 4b demonstrate that SBPD-1 selectively inhibited PSMA-expressing tumor growth in vivo. (FIG. 4a) Changes in size of PSMA+PC3 PIP and PSMA− PC3 flu subcutaneous tumors grown in NSG mice treated with varying doses of SBPD-1. (FIG. 4b) A 4-fold increase in tumor volume was scored as death of an animal;

FIG. 5 shows that SBPD-1 provided a dose-dependent survival benefit in animals with metastatic PSMA+ prostate cancer. Survival curves representing mice treated with the indicated doses of SBPD-1. Animals harbored metastatic tumors derived from PSMA+PC3/ML/PSMA cells administered intravenously;

FIG. 6a, FIG. 6b, and FIG. 6c demonstrate that SBPD-1 is not toxic to healthy mice. (FIG. 6a) Changes in weight and (FIG. 6b) survival of CD-1 mice treated with the indicated drugs. (FIG. 6c) Representative histology of selected organs after the completion of the treatment with DMSO (vehicle) and SBPD-1. (Scale bar: 100 μm). No damage occurred within tissues tested;

FIG. 7A and FIG. 7B demonstrate that PSMA expression levels in different cell lines varies. (FIG. 7A) Western Blot and (FIG. 7B) quantified band intensities (PSMA band intensities were referenced by beta actin band intensities for relative amounts comparison). Western blot analysis of PSMA in different cell lines. Cells were harvested, sonicated in PBS, and centrifuge 13200 rpm for 15 min. Collected supernatant were resolved with SDS-PAGE and transferred to nitrocellulose membrane. The membrane was blocked with 5% BSA in TBS-T and incubated overnight at 4° C. with primary antibodies (PSMA: Cell signaling, Cat #12815; Beta actin: Santa Cruz Biotechnology cat #sc47778). After incubating with secondary antibody for 1 h, membrane was visualized by chemiluminescence method using Clarity™ Western ECL Substrate (Bio Rad). Images were obtained using Gel Doc XR+ system (BIO RAD). The intensity of each band was quantified using Volume Tools in Image Lab 6.0.1 (Bio-Rad); and

FIG. 8 shows bioluminescence images of an experimental metastatic model of PSMA-expressing PC treated with SBPD-1. Treated doses are listed on top of the images.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Prostate-Specific Membrane Antigen (PSMA)-Targeted Prodrug for Selective Killing of Cells Expressing PSMA

The presently disclosed subject matter provides a novel, non-radioactive prodrug capable of selectively killing PSMA-expressing cells. The prodrug comprises a PSMA-targeted moiety, a cleavable linker, and an antineoplastic agent. In some embodiments, the PSMA-targeting moiety comprises a lysine (Lys)-Urea-glutamate (Glu) motif, Banerjee et al., 2011, which achieves high-affinity interaction with PSMA. The cleavable linker enables the release of the antineoplastic agent into lysosome via cysteine protease activities, Caculitan et al., 2017, inside the cells. In some embodiments, the antineoplastic agent comprises a cytotoxic reagent. In some embodiments, the cytotoxic agent comprises a synthetic tubulin inhibitor. In some embodiments, the synthetic tubulin inhibitor comprises monomethyl auristatin E (MMAE). Since MMAE inhibits tubulin polymerization, it will effectively kill actively dividing cancer cells while minimally affects non-dividing normal cells. MMAE exhibits very potent cytotoxicity similar to both PSMA+PC3-PIP and PSMA− PC3-flu cells.

### A. Compounds of Formula (I)

In some embodiments, the presently disclosed subject matter provides a compound comprising a PSMA-targeting moiety (T), a cleavable linker (L₁), and an antineoplastic agent (A) of formula (I):

A-L₁-T (I).

In some embodiments, the compound further comprises a non-cleavable linker (L₂), wherein the compound of formula (I) has the following general structure:

A-L₁-L₂-T (I).

In some embodiments, the compound further comprises a spacer (S), wherein the compound of formula (I) has the following general structure:

A-S-L₁-T (I).

In some embodiments, the compound further comprises a non-cleavable linker (L₂) and a spacer (S), wherein the compound of formula (I) has the following general structure:

A-S-L₁-L₂-T (I).

In certain embodiments, the PSMA-targeting moiety comprises a lysine (Lys)-Urea-glutamate (Glu)-based PSMA targeting moiety. In particular embodiments, the lysine (Lys)-Urea-glutamate (Glu)-based PSMA targeting moiety comprises:

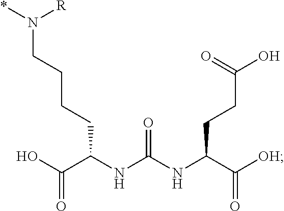

wherein * denotes a point of attachment to the non-cleavable linker (L₂) or the cleavable linker (L₁) and wherein R is H or —CH₂—R₁, wherein R₁ is selected from the group consisting of aryl substituted with one or more halogen, pyridine substituted with one or more halogen, and isoquinoline, wherein the halogen is selected from the group consisting of Cl, Br, and I

In some embodiments, R₁ is selected from the group consisting of:

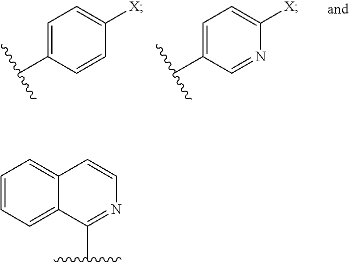

wherein each X is independently Br or I.

In certain embodiments, the non-cleavable linker is derived from disuccinimidyl suberate or polyethylene glycol (PEG). In particular embodiments, the non-cleavable linker is selected from the group consisting of: —(CH₂)ₙ and —(O—CH₂CH₂)ₘO—, wherein m and n are each independently an integer selected from the group consisting of 0, 1, 2, 3, 4, 5, 6, 7, 8, 9, 10, 11 and 12.

One of ordinary skill in the art would recognize that reagents for preparing the presently disclosed compounds of formula (I) are available in which the non-cleavable linker (L₂) is bound to the PSMA-targeting moiety (T). Accordingly, in some embodiments, a combination of the PSMA-targeting moiety (T) and the non-cleavable linker (L₂) comprises:

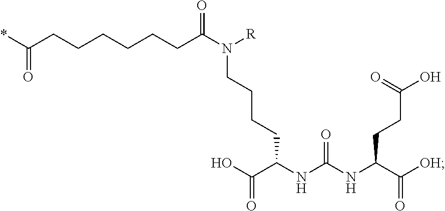

wherein * denotes a point of attachment to the cleavable linker (L₁).

In some embodiments, the cleavable linker (L₁) comprises a cathepsin-cleavable linker. In certain embodiments, the cathepsin-cleavable linker comprises one or more amino acids selected from the group consisting of valine (Val), citrulline (Cit), phenylalanine (Phe), lysine (Lys), glycine (Gly), alanine (Ala), asparagine (Asn), and combinations thereof. In particular embodiments, the cathepsin-cleavable linker comprises an amino acid combination selected from the group consisting of Val-Cit, Phe-Lys, Val-Ala, Val-Gly, Gly-Gly, Gly-Gly-Gly, and Ala-Ala-Asn. In yet more particular embodiments, the cathepsin-cleavable linker comprises Val-Cit and has the following structure:

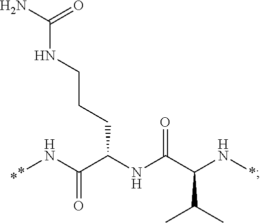

wherein * denotes a point of attachment to the PSMA-targeting moiety or a combination of the PSMA-targeting moiety (T) and the non-cleavable linker (L₂) and ** denotes a point of attachment to the antineoplastic agent (A).

In some embodiments, the spacer (S) comprises a moiety selected from the group consisting of (para-aminobenzylcarbamate) (PABC), a precursor derived from para-nitrophenol (PNP), and combinations thereof. One of ordinary skill in the art would recognize that reagents for preparing the presently disclosed compounds of formula (I) are available in which the cleavable linker (L₁) and the spacer (S) are bound to one another. Accordingly, in some embodiments, a combination of the cleavable linker (L₁) and the spacer (S) comprises:

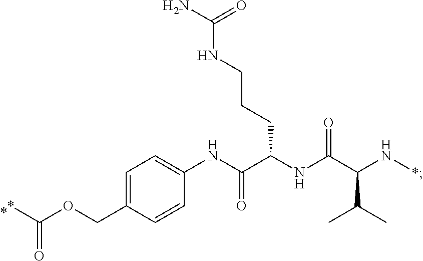

wherein * denotes a point of attachment to the PSMA-targeting moiety or a combination of the PSMA-targeting moiety (T) and the non-cleavable linker (L₂) and ** denotes a point of attachment to the antineoplastic agent (A).

In some embodiments, a combination of the cleavable linker (L₁) and the spacer (S) comprises:

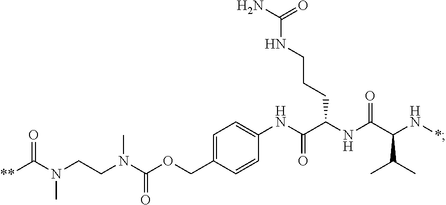

wherein * denotes a point of attachment to the PSMA-targeting moiety or a combination of the PSMA-targeting moiety (T) and the non-cleavable linker (L₂) and ** denotes a point of attachment to the antineoplastic agent (A).

In some embodiments, the antineoplastic agent comprises a cytotoxic agent. In certain embodiments, the cytotoxic agent comprises a naturally-occurring or a synthetic tubulin inhibitor. In particular embodiments, the cytotoxic agent is selected from the group consisting of monomethyl auristatin E (MMAE), monomethyl auristatin F (MMAF), monomethyl Dolastatin 10, maytansinoid, DM1, DM4, cabazitaxel, paclitaxel, and 7-ethyl-10-hydroxycamptothecin (SN-38). In some embodiments, the cytotoxic agent is selected from the group consisting of one or more topoisomerase inhibitors including, but not limited to, topoisomerase I inhibitors, such as irinotecan, topotecan, camptothecin, diflomotecan and lamellarin D, and topoisomerase II inhibitors, such as etoposide (VP-16), teniposide, doxorubicin, daunorubicin, mitoxantrone, amsacrine, ellipticines, aurintricarboxylic acid, and HU-331, a quinone synthesized from cannabidiol, and certain plant derived natural phenols, such as EGCG, genistein, quercetin, and resveratrol, which possess strong topoisomerase inhibitory properties affecting both type I and type II topoisomerase enzymes, one or more PARP inhibitors, including, but not limited to, olaparib, rucaparib, niraparib, talazoparib, veliparib, pamiparib (BGB-290), CEP 9722, E7016, iniparib (BSI 201), and 3-aminobenzamide, amatoxins, including, α-amanitin, β-amanitin, γ-amanitin, ε-amanitin, δ-amanitin, amanullin, amanullinic acid, amaninamide, amanin, and proamanullin, which, in some embodiments, are selective inhibitors of RNA polymerase II, and Pseudomonas exotoxin A.

In yet more particular embodiments, the synthetic tubulin inhibitor comprises monomethyl auristatin E (MMAE).

In particular embodiments, the compound of formula (I) comprises.

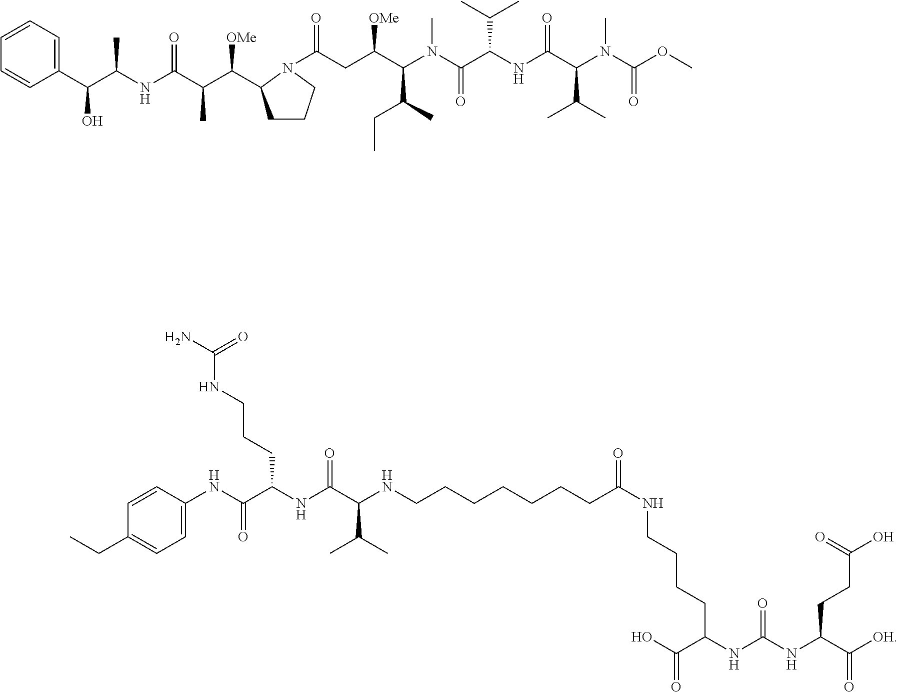

### B. Methods for Treating a PSMA-expressing Tumor or Cell

In some embodiments, the presently disclosed subject matter provides a method for treating a disease or condition associated with one or more PSMA expressing tumors or cells, the method comprising administering a therapeutically effective amount of a compound of formula (I) to a subject in need of treatment thereof.

A “tumor,” as used herein, refers to all neoplastic cell growth and proliferation, whether malignant or benign, and all precancerous and cancerous cells and tissues. In some embodiments, the tumor cells express PSMA, such as prostate tumor cells or metastasized prostate tumor cells. In other embodiments, a tumor may be treated by targeting adjacent or nearby cells which express PSMA. For example, vascular cells undergoing angiogenesis associated with a tumor may be targeted. Essentially all solid tumors express PSMA in the neovasculature. Therefore, methods of the presently disclosed subject matter can be used to treat nearly all solid tumors including, but not limited to, lung, renal cell, glioblastoma, pancreas, bladder, sarcoma, melanoma, breast, colon, germ cell, pheochromocytoma, esophageal, and stomach tumors. Also, certain benign lesions and tissues including, but not limited to, endometrium, schwannoma and Barrett's esophagus, can be imaged according to the presently disclosed methods.

In general, the “effective amount” of an active agent refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

In some embodiments, the disease or condition comprises a cancer. In certain embodiments, the cancer is selected from the group consisting of prostate cancer, renal cancer, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, adenomas, and tumor neovasculature. In particular embodiments, the cancer comprises prostate cancer. In yet more particular embodiments, the prostate cancer comprises metastatic castration-resistant prostate cancer. In other embodiments, the cancer comprises breast cancer.

A “cancer” in a subject refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and cert ain characteristic morphology and cellular markers. In some circumstances, cancer cells will be in the form of a tumor; such cells may exist locally within a subject, or circulate in the blood stream as independent cells, for example, leukemic cells.

In some embodiments, the one or more PSMA-expressing tumors or cells is in vitro, in vivo, or ex vivo. The method can be practiced in vitro or ex vivo by introducing, and preferably mixing, the compound and cell(s) or tumor(s) in a controlled environment, such as a culture dish or tube. The method can be practiced in vivo, in which case contacting means exposing at least one cell or tumor in a subject to at least one compound of the presently disclosed subject matter, such as administering the compound to a subject via any suitable route.

In particular embodiments, the one or more PSMA-expressing tumors or cells is present in a subject. The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In some embodiments, the subject is human. In other embodiments, the subject is non-human.

In some embodiments, the method further comprising administering a compound of formula (I) in combination with one or more additional cancer treatments. In certain embodiments, the one or more additional cancer treatments is selected from the group consisting of chemotherapy, radiotherapy, immunotherapy, proton therapy, photodynamic therapy, and surgery.

The term “combination” is used in its broadest sense and means that a subject is administered at least two agents, more particularly a compound of formula (I) and at least one additional therapeutic agent or cancer treatment. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds of formula (I) described herein can be administered alone or in combination with adjuvants that enhance stability of the compounds of formula (I), alone or in combination with one or more agents for treating cancer, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound of formula (I) and at least one additional therapeutic agent can be varied so long as the beneficial effects of the combination of these agents are achieved. Accordingly, the phrase “in combination with” refers to the administration of a compound of formula (I) and at least one additional therapeutic agent either simultaneously, sequentially, or a combination thereof. Therefore, a subject administered a combination of a compound of formula (I) and at least one additional therapeutic agent can receive compound of formula (I) and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound of formula (I) and at least one additional therapeutic agent are administered simultaneously, they can be administered to the subject as separate pharmaceutical compositions, each comprising either a compound of formula (I) or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound of formula (I) and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### C. Pharmaceutical Compositions and Administration

In another aspect, the present disclosure provides a pharmaceutical composition including one compound of formula (I) alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hanks' solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### Example 1

### A Prostate-Specific Membrane Antigen (PSMA)-Targeted Prodrug with a Favorable In Vivo Toxicity Profile

### 1.1 Overview

Prostate-specific membrane antigen (PSMA) is a promising target for the treatment of advanced prostate cancer (PC) and various solid tumors. Although PSMA-targeted radiopharmaceutical therapy (RPT) has enabled significant imaging and prostate-specific antigen (PSA) responses, accumulating clinical data are beginning to reveal certain limitations, including a subgroup of non-responders, relapse, radiation-induced toxicity, and the need for specialized facilities for its administration. To date non-radioactive attempts to leverage PSMA to treat PC with antibodies, nanomedicines or cell-based therapies have met with modest success. The presently disclosed subject matter provides a non-radioactive prodrug, SBPD-1, composed of a small-molecule PSMA-targeting moiety, a cancer-selective cleavable linker, and the microtubule inhibitor monomethyl auristatin E (MMAE). SBPD-1 demonstrated high binding affinity to PSMA (Kᵢ=8.84 nM) and selective cytotoxicity to PSMA-expressing PC cell lines (IC₅₀=3.90 nM). SBPD-1 demonstrated a significant survival benefit in two murine models of human PC relative to controls. The highest dose tested did not induce toxicity in immunocompetent mice. The high specific targeting ability of SBPD-1 to PSMA-expressing tumors and its favorable toxicity profile warrant its further development.

### 1.2 Background

Prostate-specific membrane antigen (PSMA) is over-expressed on the membrane of aggressive forms of prostate cancer (PC), Foss et al., 2012; Kiess et al., 2015, other human cancers, Nimmagadda et al., 2018, and endothelial cells of tumor neovasculature. Foss et al., 2012. PSMA also can also be engineered into T cells as a reporter for imaging or targeted killing. Castanares et al., 2014; Minn et al., 2019a. Those attributes have made PSMA a highly leveraged marker for imaging and targeted therapy of PSMA-expressing tumors, Huang et al., 2020; Rosenfeld et al., 2020; Petrylak et al., 2020; Machulkin et al., 2019, or cell-based therapies equipped with PSMA as a reporter. Minn et al., 2019b.

Radiopharmaceutical therapy (RPT) targeting advanced prostate cancer has been tested in clinical trials to good effect for patients who are refractory to currently approved therapies. Rowe et al., 2016; Miyahira et al., 2020; Violet et al., 2020. Despite those promising results, PSMA-targeted RPT still has limitations. RPT using beta-particle emitters, e.g., ¹⁷⁷Lu, have enabled substantial imaging and prostate-specific antigen (PSA) responses with minimal side effects, but patients tend to relapse. Miyahira, 2018; Yordanova et al., 2019. Clinical trials with alpha-particle emitters, e.g., ²²⁵Ac, have shown even better tumor responses, but also more severe toxicities including lethal renal failure in preclinical models, xerostomia, and alacrima. Kratochwil et al., 2017; Kratochwil et al., 2016; Kiess et al., 2016. Furthermore, administration of RPT requires specialized facilities for management of radioactivity. In part because of those shortcomings, PSMA-targeted therapies other than RPT are actively sought. Zhang et al., 2010; Kasten et al., 2013; Leconet et al., 2018.

The prodrug concept has been developed to avoid unwanted side effects of potent drugs with a narrow therapeutic window. Rautio et al., 2018. The prodrug itself is inactive and becomes the active pharmaceutical ingredient only through a specific interaction at the target site, such as through enzymatic cleavage of an ester or peptide bond. Although PSMA-targeted RPT has shown a measure of clinical success as noted above, an additional specificity-conferring mechanism beyond the over-expression of PSMA in malignant tissues may provide an even greater measure of safety, as PSMA is expressed in some normal tissues, notably kidney. Kinoshita et al., 2006; Silver et al., 1997. A similar concept has been tested in the form of an antibody-drug conjugate (ADC) using a humanized anti-PSMA monoclonal antibody conjugated to monomethyl auristatin E (MMAE) through a valine-citrulline linker. Wang et al., 2011; Ma, et al., 2006.

MMAE is a very potent microtubule inhibitor used for the first ADC approved by the US FDA, Brentuximab vedotin. Bartlett et al., 2014. Brentuximab vedotin used a valine-citrulline linker, Lu et al., 2016, between the drug and the antibody, which is a dipeptide designed to be enzymatically cleaved by cathepsin B, a lysosomal protease over-expressed in malignant cells. Vigneswaran et al., 2000. That PSMA ADC demonstrated a high therapeutic index in preclinical models of prostate tumors refractory to docetaxel. Wang et al., 2011. A recent phase I trial, however, revealed that despite the prodrug approach the minimal effective dose (1.8 mg/kg) was too close to the maximum tolerated dose (2.5 mg/kg) and patients suffered from neutropenia, peripheral neuropathy, and an increase in liver transaminases. Petrylak et al., 2019a. The toxicity may have been due to an unfavorable pharmacokinetic profile of the administered antibody, such as prolonged circulation, resulting in accumulation of free drug, as has been observed in clinical studies with other ADCs. Masters et al., 2018.

### 1.3 Scope of Work

A PSMA-targeted prodrug, (6S,9S,24S,28S)-1-amino-6-((4-((5S,8S,11S)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-9-isopropyl-1,8,11,18,26-pentaoxo-2,7,10,19,25,27-hexaazatriacontane-24,28,30-tricarboxylic acid (SBPD-1), was synthesized using a low-molecular-weight, urea-based PSMA targeting moiety conjugated to monomethyl auristatin E (MMAE) through a valine-citrulline linker. The target specificity, cytotoxicity against PSMA-expressing tumors, and in vivo toxicity of SBPD-1 was evaluated.

### 1.4 Results

1.4.1 SBPD-1 Binds with High Affinity to PSMA and Contains a Cathepsin B Cleavable Linker

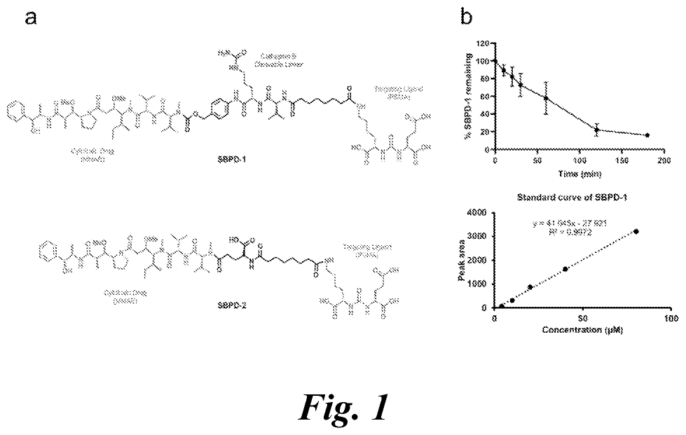

To achieve a specific, high-affinity interaction with PSMA, the low-molecular-weight (LMW) scaffold Lys-Glu-Urea-DSS originally developed in our laboratory was used. Banerjee et al., 2011. The synthetic tubulin inhibitor MMAE was conjugated to the Lys-Glu-Urea-DSS via a cathepsin B cleavable valine-citrulline linker (SBPD-1) or non-cleavable linker (SBPD-2), as a control to determine the utility of the linker (FIG. 1a). Synthesis of SBPD-1 began with known amine 1, Dubowchik et al., 2002, which on treatment with previously reported Lys-Glu-Urea-DSS, Banerjee et al., 2011, in the presence of diisopropylethylamine afforded 2 in 85% yield. Compound 2 was further converted into activated carbonate 3 in 45% yield by treating with bis(4-nitrophenyl) carbonate and subsequent reaction with MMAE, followed by deprotection to realize target conjugate SBPD-1 in 20% combined yield (Scheme 1).

Synthesis of SBPD-2 began with previously reported Lys-Glu-Urea-DSS, Banerjee et al., 2011, which on treatment with L-glutamic acid α-tert-butyl ester in the presence of diisopropylethylamine in DMF, afforded 4 in 70% yield. Compound 4 was subsequently reacted with MMAE followed by deprotection to provide target conjugate SBPD-2 in 20% combined yield (Scheme 2).

PSMA inhibitory capacity, a surrogate for affinity, was measured according to a previously described assay. Chen et al., 2008. Both conjugates, SBPD-1 and SBPD-2, demonstrated high affinity to PSMA with Kᵢ values of 8.84 nM (95% CI, 5.00-15.63) and 3.0 nM (95% CI, 1.94-4.67), respectively. Whether SBPD-1 could release MMAE when incubated with recombinant cathepsin B in vitro was tested and it was found that MMAE was efficiently released (80%) within 3 h of incubation (FIG. 1b).

### 1.4.2 SBPD-1 Selectively Kills PSMA-Expressing PC Cells In Vitro

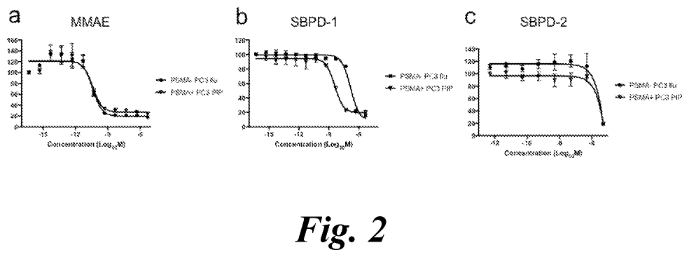

The cytotoxicity of SBPD-1 and SBPD-2 was evaluated in PSMA-expressing PC3 PIP and PSMA-negative PC3 flu cells in vitro. Mease et al., 2008; Chang et al., 1999. SBPD-1 demonstrated IC₅₀ values of 3.9 nM (95% CI, 2.8-5.5 nM) and 151.1 nM (95% CI, 104.1-219.3 nM) for PSMA+PC3 PIP and PSMA− PC3 flu cells, respectively, indicating selectivity for PSMA-expressing cells. The IC₅₀ value of 151.1 nM for PSMA− PC3 flu cells suggests release of some MMAE to enable non-selective cell kill in vitro. SBPD-2 demonstrated IC₅₀ values of 4.8 μM (95% CI, 0.8-28.5 μM) and 5.8 μM (95% CI, 0.7-47.2 μM) for PSMA+PC3 PIP and PSMA− PC3 flu cells, respectively, indicating a lack of potency regardless of PSMA expression and the need for cleavage of MMAE from the targeting moiety. MMAE alone proved exquisitely potent in both cell lines, demonstrating an IC₅₀ value of 39.2 pM (95% CI, 19.5-78.7 pM) and 40.0 pM (95% CI, 21.2-75.4 pM) for PSMA+PC3 PIP and PSMA− PC3 flu cells, respectively (FIG. 2).

### 1.4.3 SBPD-1 Selectively Kills PSMA-Expressing PC Xenografts In Vivo

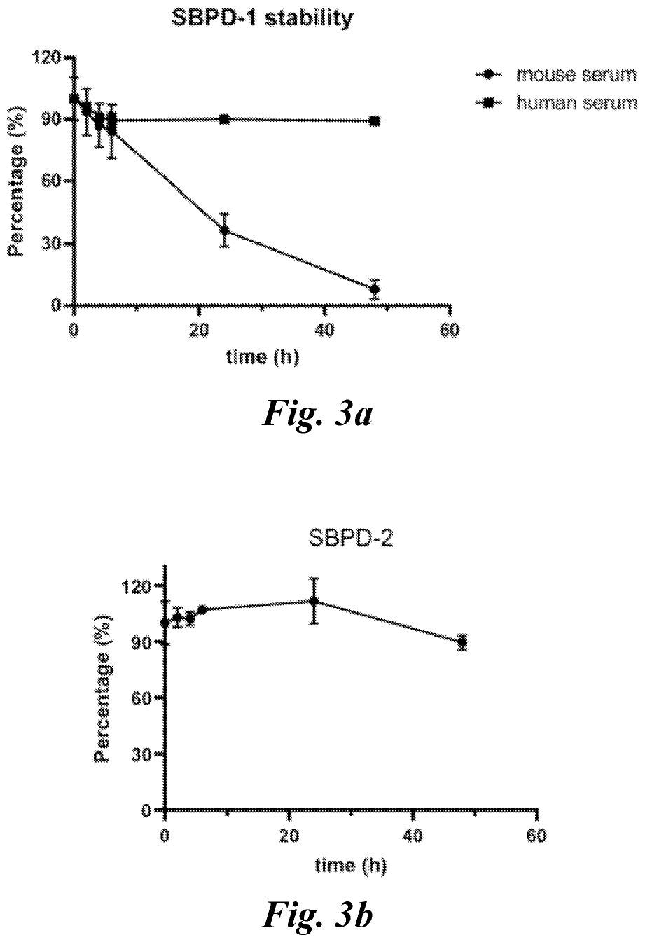

Prior to in vivo potency, the stability of SBPD-1 and SBPD-2 in murine serum was evaluated. While 90% of SBPD-2 remained intact for 48 h in murine serum (FIG. 3a), SBPD-1 was metabolized more quickly in murine serum (FIG. 3a). While more than 80% of SBPD-1 was intact in serum at 8 h of incubation, less than half represented parent compound at 24 h, and the majority of the prodrug was fully degraded by 48 h of incubation (FIG. 3a). SBPD-1, however, was stable in human serum (FIG. 3a). It has been reported that the valine-citrulline linker is stable in human and monkey serum, but that it can be hydrolyzed in mouse plasma via extracellular carboxylesterase 1c. Dorywalska et al., 2016; Anami et al., 2018. Based on those stability results, small, fractionated doses were applied for the murine efficacy study to avoid systemic toxicity that could affect the overall survival of the test animals.

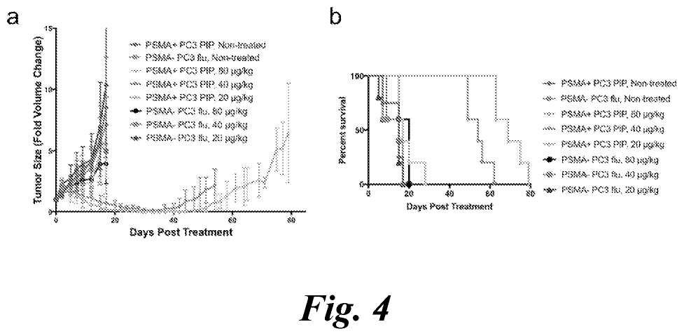

To evaluate efficacy in preclinical models of human PC, xenograft tumor models derived from PSMA+PC3 PIP and PSMA− PC3 flu cells were initially employed in NOD/SCID/IL2Rγnull (NSG) mice. Three weeks after injection of the cells, the average tumor volume reached 62.4 (±11.6) mm³, and mice were treated with 20, 40 and 80 g/kg of SBPD-1 via daily intraperitoneal (IP) injection for 30 days, n=5. Tumor growth and overall animal welfare (FIG. 4a) were monitored. Animals were scored ‘dead’ when the tumor reached 4-times its original volume (FIG. 4b). Tumors in non-treated, control mice for both tumor types, in PSMA+PC3 PIP mice treated with 20 μg/kg and in PSMA− PC3 flu mice with all three doses, grew rapidly and all animals so treated were euthanized on day 20 post-initiation of treatment (FIG. 4b). The median survival time for non-treated groups of animals harboring either PSMA+PC3 PIP or PSMA-PC3 flu tumors was 15 days. For animals harboring PSMA+PC3 PIP tumors, the median survival time of the group treated with 20 μg/kg was 17 days. The median survival times for group harboring PSMA-PC3 flu tumors treated with 20, 40, and 80 μg/kg were 15, 15, and 20 days, respectively. Doses of 40 and 80 μg/kg delivered to animals harboring PSMA+PC3 PIP tumors cleared the tumors such that they were undetectable by the completion of treatment (FIG. 4a). Approximately one week was required to be able to re-measure previously undetectable tumors in the group treated at 40 μg/kg. Two weeks were required for re-appearance of tumors in animals treated with the 80 μg/kg dose. In animals harboring PSMA+PC3 PIP tumors, both the 40 and 80 μg/kg doses provided significant survival benefits as the median survival times were 54 days [P=0.003, Log-rank (Mantel-Cox) test] and 69 days (P=0.003), respectively (FIG. 4b). Urine protein level and specific gravity measured for all test animals on Days 9 and 20 were normal, indicating that no acute renal toxicity occurred at any dose tested (Table 1).

TABLE 1
Urinalyses results from subcutaneous xenograft model.
Day 9

| | | PC3/PIP | | PC3/flu | |
|---|---|---|---|---|---|
| | Mouse | | specific | | specific |
| Treatment | # | protein | gravity | protein | gravity |
| Untreated | Mouse 1 | 30 | 1.025 | trace | 1.025 |
| | Mouse 2 | 30 | 1.025 | 30 | 1.025 |
| | Mouse 3 | ND | ND | 30 | 1.025 |
| | Mouse 4 | 30 | 1.025 | 30 | 1.025 |
| | Mouse 5 | 30 | 1.025 | 30 | 1.03 |
| SBPD-1 | Mouse 1 | trace | 1.025 | 300 | 1.025 |
| 0.08 mg/kg | Mouse 2 | 30 | 1.025 | 30 | 1.025 |
| | Mouse 3 | 30 | 1.025 | 300 | 1.025 |
| | Mouse 4 | 30 | 1.025 | 100 | 1.025 |
| | Mouse 5 | 30 | 1.025 | 30 | 1.025 |
| SBPD-1 | Mouse 1 | 30 | 1.025 | 30 | 1.025 |
| 0.04 mg/kg | Mouse 2 | trace | 1.025 | 30 | 1.025 |
| | Mouse 3 | trace | 1.025 | 30 | 1.025 |
| | Mouse 4 | 30 | 1.025 | 30 | 1.025 |
| | Mouse 5 | 30 | 1.025 | 30 | 1.025 |
| SBPD-1 | Mouse 1 | 30 | 1.025 | 30 | 1.025 |
| 0.02 mg/kg | Mouse 2 | 30 | 1.02 | 30 | 1.025 |
| | Mouse 3 | 30 | 1.025 | 30 | 1.025 |
| | Mouse 4 | 30 | 1.025 | 30 | 1.02 |
| | Mouse 5 | 30 | 1.02 | 30 | 1.025 |

Urinalyses results from subcutaneous xenograft model.
Day 20

| | | PC3/PIP | | PC3/flu | |
|---|---|---|---|---|---|
| | Mouse | | specific | | specific |
| Treatment | # | protein | gravity | protein | gravity |
| Untreated | Mouse 1 | 30 | 1.025 | trace | 1.025 |
| | Mouse 2 | 30 | 1.025 | trace | 1.03 |
| | Mouse 3 | ND | ND | 30 | 1.03 |
| | Mouse 4 | 30 | 1.025 | 30 | 1.025 |
| | Mouse 5 | 30 | 1.025 | 30 | 1.03 |
| SBPD-1 | Mouse 1 | 30 | 1.025 | 300 | 1.025 |
| 0.08 mg/kg | Mouse 2 | 30 | 1.025 | 30 | 1.025 |
| | Mouse 3 | 100 | 1.025 | 300 | 1.025 |
| | Mouse 4 | 30 | 1.025 | 30 | 1.025 |
| | Mouse 5 | 100 | 1.025 | 30 | 1.025 |
| SBPD-1 | Mouse 1 | 30 | 1.025 | 30 | 1.025 |
| 0.04 mg/kg | Mouse 2 | 30 | 1.025 | 30 | 1.03 |
| | Mouse 3 | 30 | 1.025 | ND | ND |
| | Mouse 4 | 30 | 1.025 | 30 | 1.03 |
| | Mouse 5 | 30 | 1.025 | 30 | 1.025 |
| SBPD-1 | Mouse 1 | 30 | 1.025 | 30 | 1.025 |
| 0.02 mg/kg | Mouse 2 | 30 | 1.025 | 30 | 1.01 |
| | Mouse 3 | 30 | 1.025 | 30 | 1.025 |
| | Mouse 4 | 30 | 1.025 | 30 | 1.03 |
| | Mouse 5 | 30 | 1.025 | 30 | 1.025 |

ND: Not determined.

### 1.4.4 SBPD-1 is Effective in an Experimental Metastatic Model of PSMA-Expressing PC

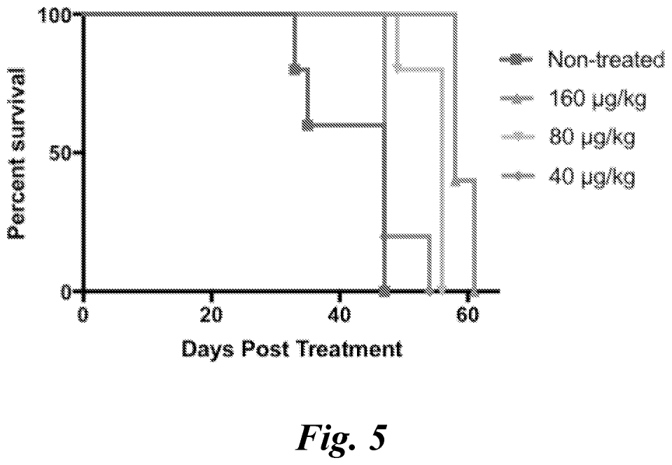

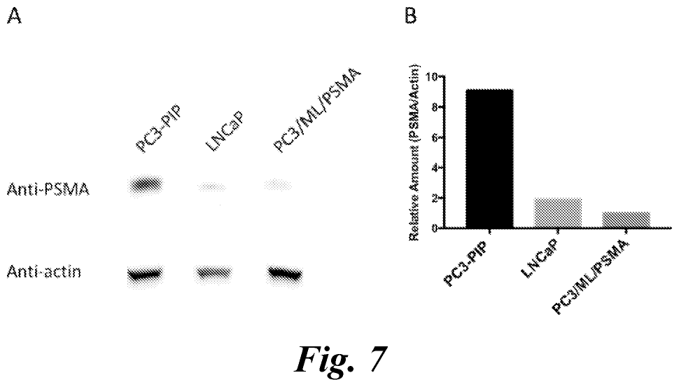

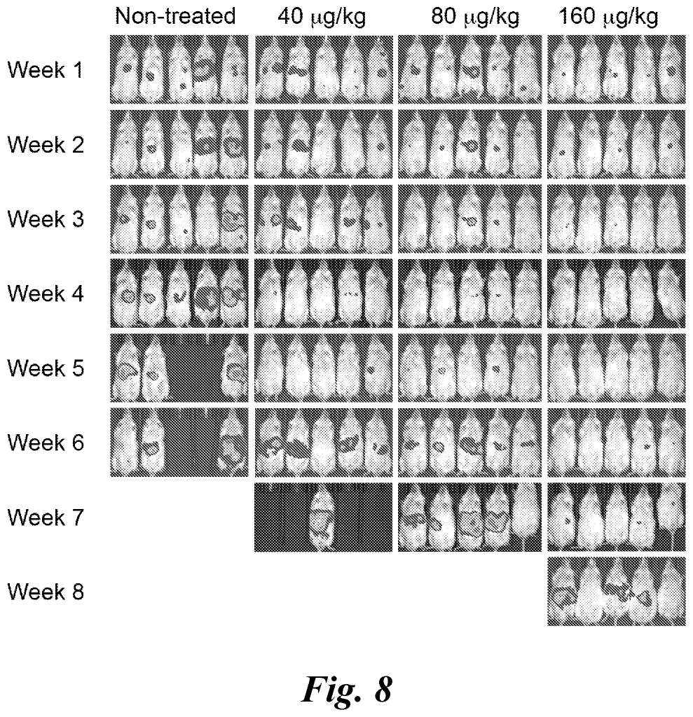

A PSMA-expressing experimental metastatic model of human PC was used to evaluate efficacy of SBPD-1 on established metastatic tumors. Kiess et al., 2016. PSMA+PC3/ML/PSMA cells were administered to NSG mice intravenously (IV) and tumors were allowed to establish for four weeks. PC3/ML/PSMA cells express firefly luciferase as an imaging reporter to allow tumor development to be monitored via weekly bioluminescence imaging (BLI). Mice were treated with 40, 80 and 160 μg/kg of SBPD-1 via daily IP injection for 30 days, n=5. The doses were increased to compensate for the lower expression of PSMA on PC3/ML/PSMA cells compared with that of PSMA+PC3 PIP tumors (FIG. 7). The 40 μg/kg dose did not show survival benefit to non-treated control mice, with median survival times of 47 days for each group. Mice treated at the 80 and 160 μg/kg dose levels, however, exhibited significant survival benefits, with median survival of 56 days [P=0.003, Log-rank (Mantel-Cox) test] and 58 days (P=0.003), respectively (FIG. 5, FIG. 8).

### 1.4.5 SBPD-1 is Non-Toxic to C57BL 6 Mice

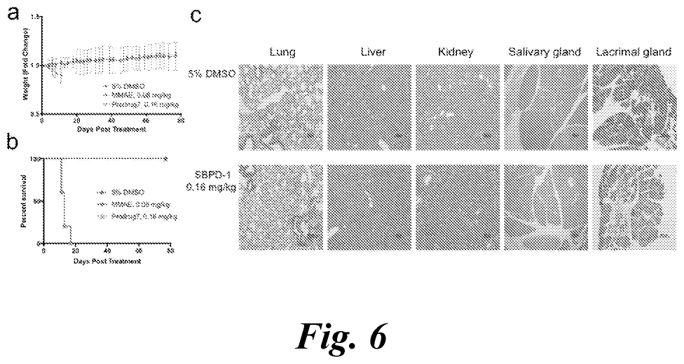

The potential toxicity of SBPD-1 was evaluated in immunocompetent animals. MMAE (80 μg/kg), SBPD-1 (160 μg/kg), and 5% DMSO were administered to healthy C57BL/6 mice (n=5). Animals were monitored for 80 days after initiation of administration. As previously reported, Qi et al., 2017, MMAE demonstrated severe toxicity as all treated mice required euthanasia during treatment due to weight loss (FIG. 6b). Mice injected with vehicle or SBPD-1 did not show any signs of toxicity and steadily gained weight (FIG. 6a). Lung, liver, kidneys, salivary and lacrimal glands were removed from all tested animals at Day 80 after initiation of SBPD-1 treatment. Histopathological examination revealed no tissue damage (FIG. 6c). Peripheral blood also was obtained from mice injected with vehicle, SBPD-1, and healthy untreated animals, and prepared serum for chemistry studies (n=5). Blood urea nitrogen (BUN), creatinine, glucose, alkaline phosphatase (ALP), total protein (T-Pro), and alanine aminotransferase (ALT) analyses showed that animals injected with either vehicle or SBPD-1 did not show differences in these values compared with those from untreated mice (Table 2).

TABLE 2
Blood chemistry for C57BL/6 mice.

| | | Test | | | | | |
|---|---|---|---|---|---|---|---|
| | | BUN | GLU | ALP | T-Pro | ALT | Cre |
| | | Unit | | | | | |
| | | mg/dl | mg/dl | IU/L | g/dl | IU/L | mg/dl |
| Untreated | Mouse 1 | 21 | 205 | 119 | 5.2 | 57 | 0.8 |
| | Mouse 2 | 27 | 211 | 162 | 5.9 | 75 | 0.8 |
| | Mouse 3 | 24 | 181 | 148 | 5.5 | 20 | 0.8 |
| | Mouse 4 | 19 | 194 | 139 | 5.9 | 31 | 0.9 |
| | Mouse 5 | 17 | 179 | 183 | 5.8 | 45 | 0.9 |
| 5% | Mouse 1 | 34 | 276 | 84 | 6.4 | 39 | 1 |
| DMSO | Mouse 2 | 35 | 205 | 81 | 6.6 | 20 | 1 |
| | Mouse 3 | 23 | 177 | 88 | 6.4 | 18 | 1 |
| | Mouse 4 | 30 | 238 | 89 | 6.5 | 32 | 0.9 |
| | Mouse 5 | 26 | 209 | 73 | 6.3 | 38 | 1 |
| SBPD-1 | Mouse 1 | 28 | 188 | 94 | 6.1 | 21 | 1 |
| 0.16 | Mouse 2 | 37 | 248 | 108 | 6.2 | 31 | 1.2 |
| mg/kg | Mouse 3 | 37 | 285 | 94 | 5.9 | 22 | 1.1 |
| | Mouse 4 | 27 | 219 | 96 | 5.9 | 14 | 1.1 |
| | Mouse 5 | 28 | 189 | 85 | 6 | 28 | 0.8 |

Complete blood counts from the mice also showed no abnormalities except for lower white blood cell count for mice injected with SBPD-1 (Table 3), which may have resulted from the relative instability of the cathepsin B linker in murine serum and subsequent bone marrow toxicity of MMAE. Dorywalska et al., 2016; Donaghy, 2016.

TABLE 3
Complete Blood Count (CBC) for C57BL/6 mice.

| | | Test | | | | | | | |
|---|---|---|---|---|---|---|---|---|---|
| | | WBC | RBC | HGB | HCT | MCV | MCH | MCHC | PLT |
| | | Unit | | | | | | | |
| | | 10³/mm³ | 10⁶/mm³ | g/dl | % | μm³ | pg | g/dl | 10³/mm³ |
| Untreated | Mouse 1 | 5.4 | 7.98 | 13.8 | 36.6 | 92 | 34.8 | 75.8 | 1072 |
| | Mouse 2 | 2.8 | 8.06 | 14.2 | 37 | 92 | 35.4 | 77 | 1062 |
| | Mouse 3 | 3.6 | 7.66 | 13.4 | 35.4 | 92 | 35 | 75.8 | 1056 |
| | Mouse 4 | 7.4 | 8.2 | 14.6 | 37.4 | 92 | 35.6 | 77.8 | 1084 |
| | Mouse 5 | 3.4 | 7.7 | 13.8 | 36 | 94 | 35.8 | 76.4 | 826 |
| 5% | Mouse 1 | 8.2 | 7.64 | 12.8 | 33.4 | 88 | 33.4 | 76.2 | 1148 |
| DMSO | Mouse 2 | 2 | 7.1 | 12 | 32 | 90 | 34 | 75.4 | 1004 |
| | Mouse 3 | 3.2 | 6.68 | 12.2 | 31.8 | 96 | 36.6 | 77 | 866 |
| | Mouse 4 | 2.4 | 8.12 | 13.4 | 36.2 | 90 | 33 | 74 | 1202 |
| | Mouse 5 | 2.4 | 6.92 | 12 | 31.6 | 92 | 34.8 | 76.2 | 994 |
| SBPD-1 | Mouse 1 | 1.6 | 8.08 | 13.4 | 36 | 90 | 33.8 | 75.8 | 1280 |
| 0.16 | Mouse 2 | 1.6 | 8.16 | 13.8 | 36.6 | 90 | 33.8 | 75.8 | 1068 |
| mg/kg | Mouse 3 | 1.8 | 7.02 | 12.4 | 32.2 | 92 | 35 | 76.4 | 1132 |
| | Mouse 4 | 1.4 | 7.98 | 13.4 | 35.6 | 90 | 33.8 | 75.8 | 1280 |
| | Mouse 5 | 2 | 8.02 | 14 | 35.8 | 90 | 34.8 | 78.2 | 1262 |

WBC: white blood cells,
RBC: red blood cells,
HGB: hemoglobin,
HCT: hematocrit,
MCV: mean corpuscular volume,
MCH: mean corpuscular hemoglobin,
MCHC: mean corpuscular hemoglobin concentration,
PLT: platelet

### 1.5 Discussion

Prostate-specific membrane antigen (PSMA) was first identified as a marker for PC through cloning of a monoclonal antibody raised against the patient-derived PC cell line, LNCaP. Horoszewicz et al., 1987. Since PSMA was discovered to be the same as the N-acetyl-L-aspartyl-L-glutamate peptidase I (NAALADase I), Carter et al., 1996, PSMA has been pursed as a target for diagnostic imaging of advanced PC with various low-molecular-weight agents. Mease et al., 2008; Pomper et al., 2002; Foss et al., 2005; Wone et al., 2006; Kularatne et al., 2009. Anti-PSMA antibodies also have been tested as PSMA-targeting entities for both molecular imaging and therapy of PC. Huang et al., 2020; Rosenfeld et al., 2020; Petrylak et al., 2020; Yao et al., 2002; Psimadas et al., 2018.

Other therapeutic approaches such as PSMA targeted-nanoparticles loaded with an anti-cancer drug, Von Hoff et al., 2016; Autio et al., 2018, or photodynamic therapy, Chen et al., 2017; Lutje et al., 2019; Wang et al., 2016, have been tested in preclinical and clinical settings. PSMA-targeted RPT has provided a new alternative to managing patients with advanced PC refractory to other therapies. Rahbar et al., 2017; Yadav et al., 2019. Recent prospective trials of ¹⁷⁷Lu-based therapies have demonstrated substantial imaging and PSA responses. Hofman et al., 2018; Aghdam et al., 2019. Fewer side effects than other systemic therapies, such as hormonal or chemotherapy, have repeatedly been shown. Fendler et al., 2017. Nevertheless, approximately 50% of patients were non-responders, and the majority of responders relapsed, requiring further cycles or other options. Yadav et al., 2019.

Questions about long-term toxicity of this method remain, particularly for α-particle emitting versions of RPT. Kratochwil et al., 2016; Kiess et al., 2016; Kratochwil et al., 2018a; Kratochwil et al., 2018b. Although PSMA-targeted RPT is promising and fraught with fewer adverse events compared to the conventional cytotoxic therapies, radiation exposure to normal organs can result in xerostomia or other off-target effects. Kratochwil et al., 2017; Kratochwil et al., 2016; Kiess et al., 2016; Kratochwil et al., 2018a; Kratochwil et al., 2018b.

A PSMA-targeted prodrug equipped with additional specificity to malignant cells may provide an enhanced therapeutic index. Several PSMA-targeted prodrugs were tested in both preclinical and clinical settings. Kularatne et al., 2010, tested various cytotoxic drugs as a form of prodrug by conjugating them to the PSMA-targeted agent, 2-[3-(1, 3-dicarboxy propyl)ureido] pentanedioic acid.

Those prodrugs utilized a disulfide linker to enable drug release in the reducing environment of the cytoplasm. Some of the tested drugs exhibited cytotoxicity to PSMA-expressing LNCaP cells at single- or double-digit nanomolar concentration levels. In vivo safety and efficacy of those drugs, however, have not been tested. Mipsagargin (G-202) is a prodrug consisting of an analog of thapsigargin conjugated to a PSMA-cleavable peptide. Denmeade et al., 2012. Thapsigargin is a potent inhibitor of the sarcoplasmic/endoplasmic reticulum calcium adenosine triphosphatase (SERCA) pump essential for cell viability. Mipsagargin was used to target the PSMA-expressing tumor neovasculature of various solid cancers. Despite promising preclinical and phase I results, Denmeade et al., 2012, phase II trials showed no clinical benefit for advanced hepatocellular carcinoma. Mahalingam et al., 2019. A PSMA-targeted antibody-MMAE conjugate (ADC) has been tested and showed favorable preclinical efficacy. Wang et al., 2011; Ma et al., 2006.

In a phase I trial with that conjugate, however, the therapeutic window proved narrow, necessitating modification of dose selection if the compound were to advance further. Petrylak et al., 2019a. The authors of that trial hypothesized that the toxicity may have been due to the systemic concentration of free MMAE released from the antibody. Petrylak et al., 2019a. The results from the corresponding phase II trial were recently published. Petrylak et al., 2019b. Toxicity was noted shortly after the initiation of the trial such that a dose reduction was necessary for it to continue. A partial radiologic response was obtained in only 2 of 119 participants, with none reporting a complete response. Petrylak et al., 2019b.

Note that PSMA+PC3 PIP cells were used to generate subcutaneous tumors that may not precisely reflect the case as it may occur in patients. Although the number of PSMA molecules per PSMA+PC3 PIP cell were not measured in the current study, it has been previously shown there to be an order of magnitude higher PSMA expression in these cells than in LNCaP cells, which are patient-derived. Kiess et al., 2016. PSMA+PC3/ML/PSMA cells used for the metastatic model, however, have comparable PSMA expression to that of LNCaP cells. Nevertheless, the PSMA+PC3 PIP/PSMA− PC3 flu cells were used to generate subcutaneous tumors to minimize the number of variables between cells used, as these lines are otherwise isogenic, and to see if any signal could be obtained in this proof-of-principal study.

SBPD-1 was designed for safe delivery of the potent toxin MMAE to maximize its therapeutic index. There are three layers of specificity of this agent for malignant cells. First, there is high-affinity, specific PSMA targeting followed by internalization of drug-bound PSMA. Notably PSMA tends to localize to the centrosome upon internalization, Kiess et al., 2015, enabling it to deliver a drug that interrupts microtubule formation to the compartment in which it can be most effective. Second, MMAE is released only upon enzymatic cleavage by cathepsin B, which is upregulated in the lysosomes of cancer cells. Vigneswaran et al., 2000. The same drug with non-cleavable linker (SBPD-2) showed about 7,100-fold less potency in PSMA+ cancer cells (FIG. 2). Third, MMAE inhibits microtubule polymerization, an essential process for cell division of cancer cells. A further advantage of the small-molecule approach is that drug conjugates tend to have superior tumor penetration and more rapid clearance from non-target sites than do ADCs. Ovacik and Lin, 2018.

Since prior reports, Dorywalska et al., 2016, Anami et al, 2018, as well as the present results (FIG. 3a) have suggested that the valine-citrulline linker is unstable in murine serum, the dosing plan was modified to consist of several fractionated doses. In vivo safety results with an immunocompetent murine model showed no toxicity with the highest doses tested in the efficacy study (FIG. 6, Table 2). It is likely that a clinical dosing plan could consist of less frequent administration as the valine-citrulline linker has been reported to be stable in human plasma. Anami et al, 2018.

In summary, a low-molecular-weight, PSMA-targeted prodrug has been generated and tested that demonstrated tumor penetration and specificity sufficient to provide survival differences between PSMA+ tumor-bearing animals and animals bearing isogenic tumors devoid of PSMA, including in a metastatic model. Furthermore, despite carrying the potent anti-tumor agent MMAE, the conjugate was non-toxic. It is thought that lower toxicity was due to the controlled environment to which MMAE was delivered, by virtue of the presence of a cathepsin B cleavable linker in the molecule. Compounds of this class or those employing similar strategies may enable safe and effective targeting of PSMA-expressing lesions in patients.

### 1.6 Methods

### 1.6.1 General Methods and Materials for Syntheses of Prodrugs

Detailed methods for the syntheses of prodrugs are described in Section 1.7 herein below. Commercially available reagents and solvents for syntheses were analytical grade and used without further purification. Diisopropylethylamine (DIPEA), trifluoracetic acid (TFA), 4-(Dimethyl amino) pyridine (DMAP), pyridine (Py) and N-(3-dimethylaminopropyl)-N-ethylcarbodiimide (EDC) were purchased from Sigma-Aldrich (Allentown, PA, USA). L-Glutamic acid 5-tert-butyl ester, bis(4-nitrophenyl) carbonate and 1-hydroxybenzotriazole hydrate (HOBt) were purchased from Chem-Impex International (Wood Dale, IL, USA), disuccinimidyl suberate was purchased from TCI America (Pittsburgh, PA, USA) and monomethyl auristatin E (MMAE) was purchased from BroadPharm (San Diego, CA, USA). High performance liquid chromatographic (HPLC) purification of final compounds (SBPD-1 and SBPD-2) was performed using a C₁₈ Luna 10 mm×250 mm column (Phenomenex, Torrance, CA, USA) on an Agilent 1260 infinity LC system (Santa Clara, CA, USA) and eluted with water (0.1% TFA) (A) and CH₃CN (0.1% TFA) (B). ¹H NMR spectra were recorded on a Bruker Ultrashield™ 500 MHz spectrometer. Chemical shifts (6) are reported in parts per million (ppm) downfield by reference to proton resonances resulting from incomplete deuteration of the NMR solvent and the coupling constants (J) was reported in Hertz (Hz). High resolution mass spectra were obtained by the University of Notre Dame Mass Spectrometry and Proteomics Facility, Notre Dame, IN using ESI by direct infusion on a Bruker micrOTOF-II.

### 1.6.2 Cathepsin B Cleavage

Release of MMAE from prodrugs by a recombinant cathepsin B was analyzed using a modified method from previously published work. Dubowchik et al., 2002. Prodrug stock solutions (8 μL, 10 mM) were added to the 2 mL cathepsin B (MilliporeSigma, Cat #C8571, Burlington, MA, USA) containing buffer (25 mM acetate, 1 mM EDTA, pH 5, pre-warmed at 37° C.) at the final concentration of 30 nM (cathepsin B) and 40 μM (prodrug). Aliquots (200 μL) were periodically removed and enzymatic activity was stopped by the addition of thioprotease inhibitor E-64 (30 nM in the final solution, MilliporeSigma, Cat #E3132). The samples were centrifuged and the supernatants were analyzed by HPLC (Waters 600 E coupled with Varian prostar detector, Milford, MA, USA). Samples were prepared at 0, 10, 20, 30, 60, 90, and 120 min. Experiments were performed in triplicate.

### 1.6.3 PSMA Affinity and In Vitro Cytotoxicity

PSMA affinities of SBPD-land SBPD-2 were measured using the modified Amplex™ Red glutamic acid/glutamate oxidase assay as previously described. Chen et al., 2008. PSMA-expressing PC3-PIP, PSMA-negative PC3-flu, PSMA-positive PC3/ML/PSMA and PSMA-negative PC3/ML were maintained as previously described. Kiess et al., 2016. One thousand cells (PC3-PIP or PC3-flu) were seeded in 96 well plates 24 h prior to drug treatment. Drug was added to each well in serial dilution and incubated for 24, 48 or 72 hr. Cell viability was measured using TACS XTT Cell Proliferation Assay (Trevigen, Cat #4891-25-K, Gaithersburg, MD) at each time point according to the manufacturer's protocol. IC₅₀ values were calculated using GraphPad Prism 7 software.

### 1.6.4 Serum Stability

Serum stability of prodrugs was analyzed using a modified method from previously published work. Chu et al., 2012. Prodrug stock solution (8 μL, 10 mM) was incubated with 100% mouse serum (final concentration of the serum was 80% after the mixing with prodrug solution) at 37° C. Aliquots (50 μL) were periodically removed at 2, 4, 8, 24, and 48 h, and diluted with cold ice CH₃OH (100 μL) to precipitate proteins. The samples were centrifuged, and the supernatants were analyzed by HPLC. Stability was calculated based on the peak area of the prodrug at each time point. Experiments were performed in triplicate.

### 1.6.5 Preclinical Evaluation of SBPD-1

Animal studies were performed under the guidance of a protocol approved by the Johns Hopkins Animal Care and Use Committee. NSG (NOD/SCID/IL2Rγnull) mice were purchased from the Johns Hopkins University Sydney Kimmel Comprehensive Cancer Center Animal Resources Core. C57BL/6 mice were purchased from Jackson Laboratory (Bar Harbor, ME, USA). NSG mice were injected with 1.5 million PC3/PIP or 1 million PC3/flu cells at the lower left flank. Two weeks after the injection of cells, mice were treated with 20, 40, 80 μg/kg of SBPD-1 formulated in 100 μL of sterile saline via daily intraperitoneal (IP) injection for 30 days. Tumor volumes were measure twice per week. Urinalysis was performed using URS-10 Urine Reagent Strips (LW Scientific Inc. Lawrenceville, GA).

For the metastatic model, NSG mice were injected with 0.75 million PC3/ML/PSMA cells via the tail vein. Four weeks after the injection mice were treated with 40, 80, 160 μg/kg of SBPD-1 formulated in 100 μL of sterile water via daily intraperitoneal injection for 30 days. BLI was performed weekly using the IVIS Spectrum in vivo imaging system (Perkin Elmer, Waltham, MA).

### 1.6.6 In Vivo Toxicity

Male C57BL/6 mice were purchased from Jackson Laboratory. Ten-week-old mice were injected with the indicated doses of MMAE (formulated in 5% DMSO), SBPD-1 (formulated in saline) or 5% DMSO intraperitoneally (daily for 30 days, n=5). Animals were monitored daily for weight changes and other abnormalities for 80 days. Animals were euthanized in a CO₂ chamber at day 80, and blood, lung, liver, kidney, salivary gland, and lacrimal gland were collected for complete blood counts, blood chemistry, and histopathological analyses. Complete blood counts including white blood cells (WBC), red blood cells (RBC), hemoglobin (HGB), hematocrit (HCT), mean corpuscular volume (MCV), mean corpuscular hemoglobin (MCH), mean corpuscular hemoglobin concentration (MCHC), and platelet (PLT) were measured using scil Vet ABC™ Hematology Analyzer (scil animal care company, Gurnee, IL). Blood chemistry parameters including blood urea nitrogen (BUN), glucose (GLU), Alkaline Phosphatase (ALP), total protein (T-Pro), Alanine aminotransferase (ALT) and Creatinine (Cre) were measured with Spotchem EZ chemistry analyzer (Arkray USA, Edina, MN). Hematoxylin and eosin slides were generated for five organs and examined by certified veterinary pathologist.

### 1.7 Experimental Procedures and Spectral Characterization

### 1.7.1 Synthesis of SBPD-1

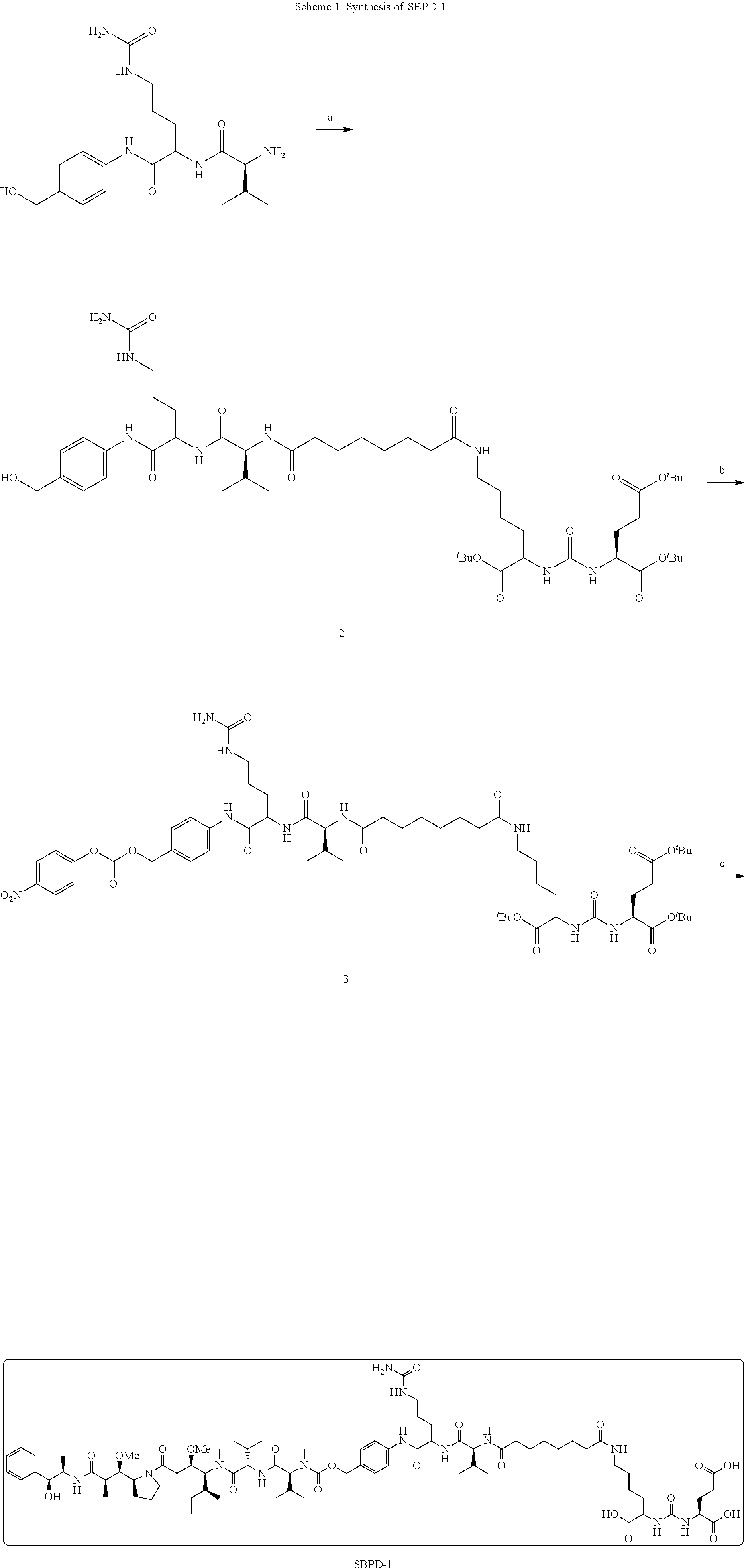

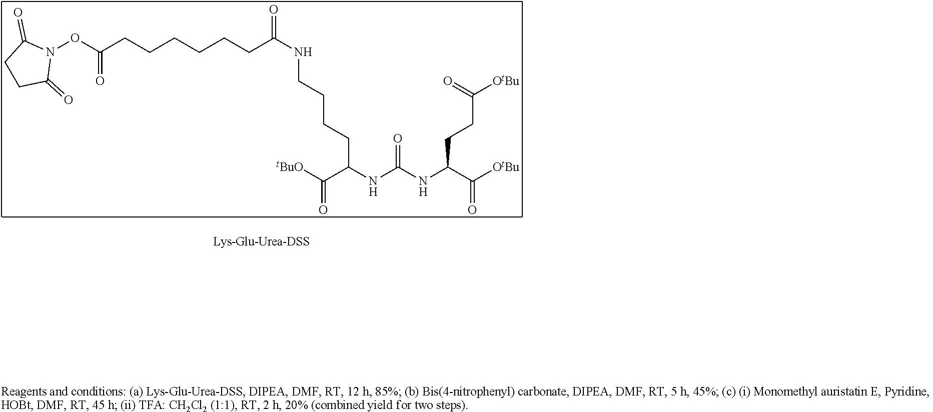

Tri-tert-butyl (6S,9S,24S,28S)-1-amino-6-((4-(hydroxymethyl)phenyl)carbamoyl)-9-isopropyl-1,8,11,18,26-pentaoxo-2,7,10,19,25,27-hexaazatriacontane-24,28,30-tricarboxylate (2): To a stirred solution of amine 1 (38 mg, 0.1 mmol, 1.0 eq.) and di-tert-butyl (((S)-1-(tert-butoxy)-6-(8-((2,5-dioxopyrrolidin-1-yl)oxy)-8-oxooctanamido)-1-oxohexan-2-yl)carbamoyl)-L-glutamate (74 mg, 0.1 mmol, 1.0 eq.) in dimethylformamide (1.0 mL) was added diisopropylethylamine (52 μL, 0.3 mmol, 3.0 eq.) at room temperature. The resulted mixture was stirred for 12 h at room temperature and concentrated in vacuo. The thick residue was triturated with diethyl ether (20 mL) for 30 minutes, and the solid was collected by filtration and washed with diethyl ether to obtain compound 2 as a colorless solid (85.5 mg, 85%). H¹—NMR (500 MHz, DMSO-d₆): δ 9.89 (s, 1H), 8.05 (d, J=7.5 Hz, 1H), 7.81 (d, J=8.5 Hz, 1H), 7.77-7.69 (m, 1H), 7.53 (d, J=8.0 Hz, 2H), 7.22 (d, J=8.0 Hz, 2H), 6.35-6.21 (m, 2H), 6.05-5.96 (m, 1H), 5.41 (s, 2H), 5.13 (t, J=5.0 Hz, 1H), 4.42 (d, J=4.5 Hz, 2H), 4.38-4.31 (m, 1H), 4.17 (t, J=7.5 Hz, 1H), 4.06-3.97 (m, 1H), 3.98-3.90 (m, 1H), 3.15-3.07 (m, 2H), 3.06-2.95 (m, 2H), 2.95-2.91 (m, 1H), 2.31-2.08 (m, 4H), 2.06-1.79 (m, 4H), 1.74-1.52 (m, 4H), 1.53-1.30 (m, 14H), 1.38 (s, 27H), 0.83 (dd, J=6.5, 7.5 Hz, 6H); HRMS (ESI) m/z: [M+H]+ calcd for C₅₀H₈₅N₈O₁₃, 1005.6226; found, 1005.6230.

Tri-tert-butyl (6S,9S,24S,28S)-1-amino-9-isopropyl-6-((4-((((4-nitrophenoxy) carbonyl)oxy) methyl)phenyl)carbamoyl)-1,8,11,18,26-pentaoxo-2,7,10,19,25,27-hexaazatriacontane-24,28,30-tricarboxylate (3): To a stirred solution of compound 2 (43 mg, 0.042 mmol, 1.0 eq.) and bis(4-nitrophenyl) carbonate (26 mg, 0.085 mmol, 2.0 eq.) in dimethylformamide (1.5 mL) was added diisopropylethylamine (20 μL, 0.085 mmol, 3.0 eq.) at room temperature under nitrogen atmosphere. The resulted mixture was stirred for 5 h at room temperature and concentrated in vacuo. The residue was triturated with diethyl ether (20 mL) for 1 h, and the solid was collected by filtration and washed with diethyl ether to obtain compound 3 as a brown color solid (22.5 mg, 45%). H¹-NMR (500 MHz, DMSO-d₆): δ 10.05 (s, 1H), 8.30 (d, J=9.0 Hz, 2H), 8.15-8.06 (m, 1H), 7.80 (d, J=8.0 Hz, 1H), 7.73 (t, J=5.0 Hz, 1H), 7.64 (d, J=8.5 Hz, 2H), 7.55 (d, J=9.5 Hz, 2H), 7.40 (d, J=8.5 Hz, 2H), 6.27 (dd, J=8.5, 16.5 Hz, 2H), 6.05-5.94 (m, 1H), 5.42 (s, 2H), 5.23 (s, 2H), 4.40-4.32 (m, 1H), 4.22-4.13 (m, 1H), 4.06-3.98 (m, 1H), 3.97-3.90 (m, 1H), 3.09-2.88 (m, 5H), 2.28-2.06 (m, 4H), 2.05-1.80 (m, 4H), 1.75-1.54 (m, 4H), 1.54-1.41 (m, 8H), 1.37 (s, 27H), 1.29-1.14 (m, 6H), 0.90-0.77 (m, 6H); HRMS (ESI) m/z: [M+H]+ calcd for C₅₇H₈₈N₉O₁₇, 1170.6264; found, 1170.6292.

(6S,9S,24S,28S)-1-Amino-6-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-9-isopropyl-1,8,11,18,26-pentaoxo-2,7,10,19,25,27-hexaazatriacontane-24,28,30-tricarboxylic acid (SBPD-1): To a stirred solution of compound 3 (19.9 mg, 0.0169 mmol) and MMAE (9.0 mg, 0.0125 mmol) in dimethylformamide (800 μL) was added 1-hydroxybenzotriazole hydrate (0.33 mg, 0.0025 mmol) and pyridine (200 μL) at room temperature. The resulted mixture was stirred for 48 h at room temperature and concentrated in vacuo. The obtained residue was dissolved in 50% TFA in dichloromethane (2 mL) and resulted mixture was stirred at room temperature for 2 h followed by concentrated in vacuo. The crude was purified by preparative RP-HPLC chromatography using 0.1% TFA in H₂O and 0.10% TFA in acetonitrile as eluents followed by lyophilization afforded target compound (SBPD-1) as a colorless solid (5.3 mg, 20%). [RP-HPLC purification was achieved using Agilent System, λ 254 nm, 250 mm×10 mm Phenomenex Luna Cis column, solvent gradient: 80% H₂O (0.1% TFA) and 20% ACN (0.1% TFA), reaching 60% of ACN in 20 min at a flow rate of 10 m/min, product eluted at 14.4 min]. H¹-NMR (500 MHz, DMSO-d₆): δ 9.98 (s, 1H), 8.33-7.99 (m, 1H), 8.09 (d, J=7.0 Hz, 1H), 7.93-7.52 (m, 5H), 7.39-7.11 (m, 7H), 6.30 (dd, J=8.5, 16.5 Hz, 2H), 5.99 (s, 1H), 5.55-5.29 (m, 2H), 5.13-4.91 (m, 2H), 4.67-2.78 (m, 32H, merged in moisture peak), 2.43-1.87 (m, 12H), 1.85-1.15 (m, 25H), 1.09-0.68 (m, 31H); HRMS (ESI) m/z: [M+Na]+ calcd for C₇₈H₁₂₅N₁₃NaO₂₁, 1602.9009; found, 1602.9005.

### 1.7.2 Synthesis of SBPD-2

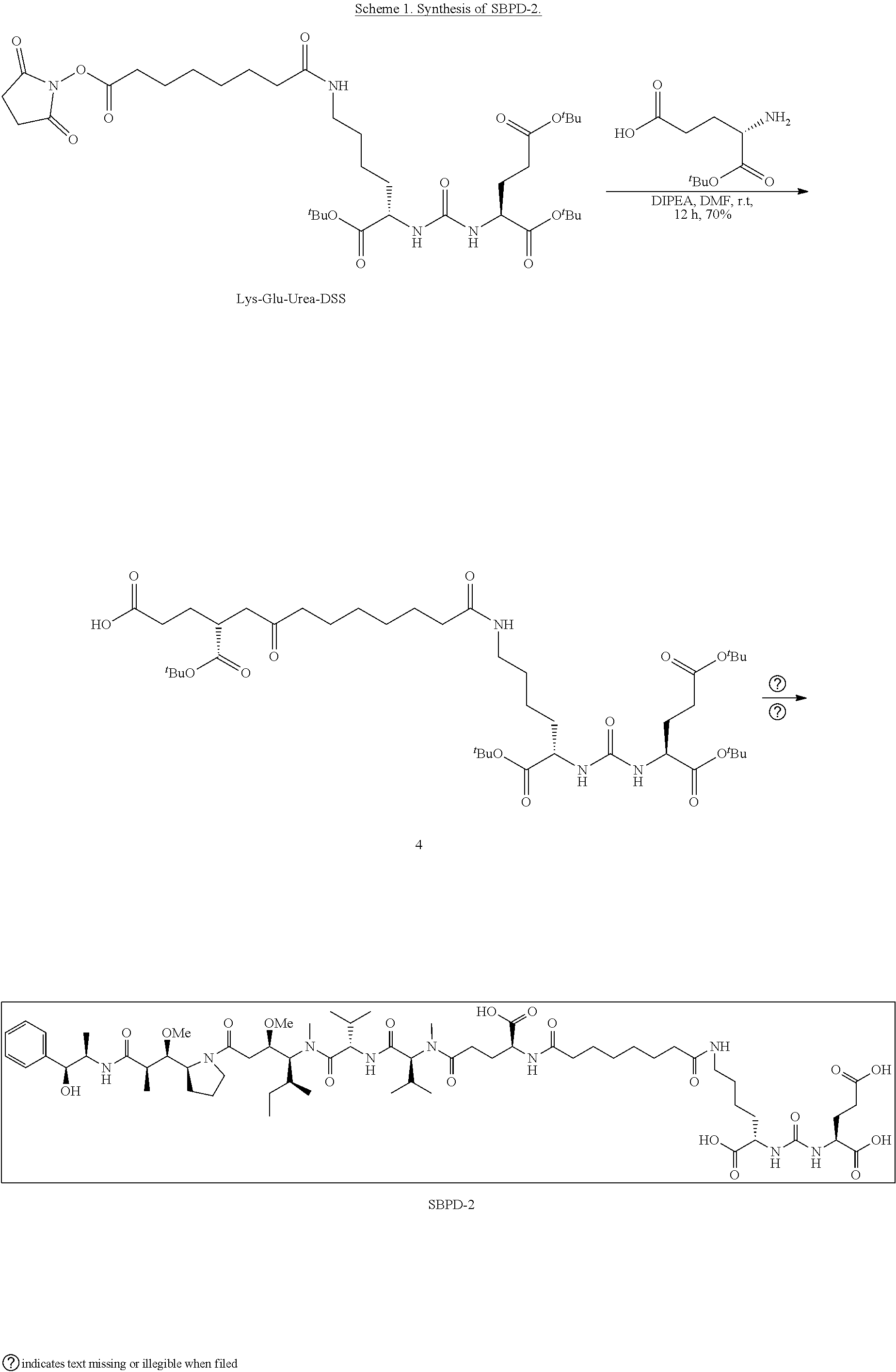

(7S,11S,26R)-7,26-Bis(tert-butoxycarbonyl)-11-carboxy-2,2-dimethyl-4,9,17,24-tetraoxo-3-oxa-8,10,16,25-tetraazanonacosan-29-oic acid (4): To a stirred solution of (S)-4-amino-5-(tert-butoxy)-5-oxopentanoic acid (41 mg, 0.202 mmol, 1.5 eq) and di-tert-butyl (((S)-1-(tert-butoxy)-6-(8-((2,5-dioxopyrrolidin-1-yl)oxy)-8-oxooctanamido)-1-oxohexan-2-yl)carbamoyl)-L-glutamate (100 mg, 0.135 mmol, 1.0 eq) in dimethyl formamide (1.0 mL) was added diisopropylethylamine (95 μL, 0.539 mmol, 4.0 eq) at room temperature. The resulted mixture was stirred for 12 h at room temperature and concentrated in vacuo. The residue was purified by using ACN/H₂O on C₁₈ Sep-Pak column to provide compound 4 (78 mg, 70%) as a colorless solid. H¹—NMR (500 MHz, CDCl₃): δ 6.97-6.88 (m, 1H), 6.78 (d, J=8.0 Hz, 1H), 6.02-5.70 (m, 2H), 4.53-4.44 (m, 1H), 4.34-4.17 (m, 2H), 3.33-3.12 (m, 2H), 2.50-2.14 (m, 8H), 2.09-1.93 (m, 2H), 1.87-1.70 (m, 2H), 1.70-1.25 (m, 14H), 1.45 (s, 9H), 1.44 (s, 9H), 1.43 (s, 9H), 1.41 (s, 9H).

(4S,7S,10S,15S,30S,34S)-4-((S)-Sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12,17,24,32-hexaoxo-2-oxa-5,8,11,16,25,31,33-heptaazahexatriacontane-15,30,34,36-tetracarboxylic acid (SBPD-2): To a stirred solution of compound 4 (9.5 mg, 0.0114 mmol) and MMAE (8.2 mg, 0.0114 mmol) in dichloromethane (1.0 mL) was added HOBt (1.7 mg, 0.0125 mmol), EDC (2.41 mg, 0.0125 mmol) and DIPEA (6 μL, 0.0343 mmol) at room temperature. The resulted mixture was stirred for 3 days at room temperature and concentrated in vacuo. The obtained residue was dissolved in 50% TFA in dichloromethane (2 mL) and resulted mixture was stirred at room temperature for 1 h followed by concentrated in vacuo. The crude was purified by preparative RP-HPLC chromatography using 0.10% TFA in H₂O and 0.10% TFA in acetonitrile as eluents followed by lyophilization afforded target compound (SBPD-2) as a colorless solid (3 mg, 20%). [RP-HPLC purification was achieved using Agilent System, λ 220 nm, 250 mm×10 mm Phenomenex Luna C₁₈ column, solvent gradient: 90% H₂O (0.1% TFA) and 10% ACN (0.1% TFA), reaching 60% of ACN in 20 min at a flow rate of 10 mL/min, product eluted at 14.2 min]. H¹—NMR (500 MHz, DMSO-d₆): (13.20-11.72 (bs, 3H), 8.59-8.49 (m, 0.5H), 8.10-7.96 (m, 1H), 7.93-7.77 (m, 1H), 7.71 (t, J=5.0 Hz, 1H), 7.62 (d, J=8.0 Hz, 0.5H), 7.35-7.22 (m, 4H), 7.20-7.13 (m, 1H), 6.36-6.24 (m, 2H), 4.79-4.60 (m, 1H), 4.53-4.38 (m, 2H), 4.27-3.94 (m, 7H), 3.28-3.07 (m, 10H), 3.02-2.79 (m, 6H), 2.43-2.21 (m, 7H), 2.13-1.99 (m, 7H), 1.92-1.60 (m, 9H), 1.53-1.16 (17H), 1.07-0.97 (m, 6H), 0.87-0.68 (m, 18H); HRMS (ESI) m/z: [M+H]+ calcd for C₆₅H₁₀₈N₈O₁₉, 1304.7735; found, 1304.7725.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Foss, C. A., Mease, R. C., Cho, S. Y., Kim, H. J. & Pomper, M. G. GCPII imaging and cancer. Curr Med Chem 19, 1346-1359 (2012).
Kiess, A. P. et al. Prostate-specific membrane antigen as a target for cancer imaging and therapy. Q J Nucl Med Mol Imaging 59, 241-268 (2015).
Nimmagadda, S. et al. Low-Level Endogenous PSMA Expression in Nonprostatic Tumor Xenografts Is Sufficient for In Vivo Tumor Targeting and Imaging. J Nucl Med 59, 486-493, doi:10.2967/jnumed.117.191221 (2018).
Castanares, M. A. et al. Evaluation of prostate-specific membrane antigen as an imaging reporter. J Nucl Med 55, 805-811, doi:10.2967/jnumed.113.134031 (2014).
Minn, I. et al. Imaging CAR T cell therapy with PSMA-targeted positron emission tomography. Sci Adv 5, eaaw5096, doi:10.1126/sciadv.aaw5096 (2019a).
Huang, C. T. et al. Development of 5D3-DM1: A Novel Anti-Prostate-Specific Membrane Antigen Antibody-Drug Conjugate for PSMA-Positive Prostate Cancer Therapy. Mol Pharm, doi:10.1021/acs.molpharmaceut.0c00457 (2020).
Rosenfeld, L. et al. Nanobodies Targeting Prostate-Specific Membrane Antigen for the Imaging and Therapy of Prostate Cancer. J Med Chem 63, 7601-7615, doi:10.1021/acs.jmedchem.0c00418 (2020).
Petrylak, D. P. et al. PSMA ADC monotherapy in patients with progressive metastatic castration-resistant prostate cancer following abiraterone and/or enzalutamide: Efficacy and safety in open-label single-arm phase 2 study. Prostate 80, 99-108, doi:10.1002/pros.23922 (2020).
Machulkin, A. E. et al. Synthesis and biological evaluation of PSMA-targeting paclitaxel conjugates. Bioorg Med Chem Lett 29, 2229-2235, doi:10.1016/j.bmcl.2019.06.035 (2019).
Minn, I., Rowe, S. P. & Pomper, M. G. Enhancing CAR T-cell therapy through cellular imaging and radiotherapy. Lancet Oncol 20, e443-e451, doi:10.1016/51470-2045(19)30461-9 (2019b).
Rowe, S. P. et al. Prostate-Specific Membrane Antigen-Targeted Radiohalogenated PET and Therapeutic Agents for Prostate Cancer. J Nucl Med 57, 90S-96S, doi:10.2967/jnumed.115.170175 (2016).
Miyahira, A. K. et al. Meeting report from the Prostate Cancer Foundation PSMA theranostics state of the science meeting. Prostate, doi:10.1002/pros.24056 (2020).
Violet, J. et al. Long-Term Follow-up and Outcomes of Retreatment in an Expanded 50-Patient Single-Center Phase II Prospective Trial of (177)Lu-PSMA-617 Theranostics in Metastatic Castration-Resistant Prostate Cancer. J Nucl Med 61, 857-865, doi: 10.2967/jnumed.119.236414 (2020).
Miyahira, A. K. et al. Meeting report from the Prostate Cancer Foundation PSMA-directed radionuclide scientific working group. Prostate 78, 775-789, doi:10.1002/pros.23642 (2018).
Yordanova, A. et al. Outcome and safety of rechallenge [(177)Lu]Lu-PSMA-617 in patients with metastatic prostate cancer. Eur J Nucl Med Mol Imaging 46, 1073-1080, doi:10.1007/s00259-018-4222-x (2019).
Kratochwil, C. et al. Targeted alpha-Therapy of Metastatic Castration-Resistant Prostate Cancer with (225)Ac-PSMA-617: Dosimetry Estimate and Empiric Dose Finding. J Nucl Med 58, 1624-1631, doi:10.2967/jnumed.117.191395 (2017).
Kratochwil, C. et al. 225Ac-PSMA-617 for PSMA-Targeted alpha-Radiation Therapy of Metastatic Castration-Resistant Prostate Cancer. J Nucl Med 57, 1941-1944, doi:10.2967/jnumed.116.178673 (2016).
Kiess, A. P. et al. (2S)-2-(3-(1-Carboxy-5-(4-211At-Astatobenzamido)Pentyl)Ureido)-Pentanedioic Acid for PSMA-Targeted alpha-Particle Radiopharmaceutical Therapy. J Nucl Med 57, 1569-1575, doi:10.2967/jnumed.116.174300 (2016).
Zhang, A. X. et al. A remote arene-binding site on prostate specific membrane antigen revealed by antibody-recruiting small molecules. J Am Chem Soc 132, 12711-12716, doi:10.1021/ja104591m (2010).
Kasten, B. B., Liu, T., Nedrow-Byers, J. R., Benny, P. D. & Berkman, C. E. Targeting prostate cancer cells with PSMA inhibitor-guided gold nanoparticles. Bioorg Med Chem Lett 23, 565-568, doi:10.1016/j.bmcl.2012.11.015 (2013).
Leconet, W. et al. Anti-PSMA/CD3 Bispecific Antibody Delivery and Antitumor Activity Using a Polymeric Depot Formulation. Mol Cancer Ther 17, 1927-1940, doi:10.1158/1535-7163.MCT-17-1138 (2018).
Rautio, J., Meanwell, N. A., Di, L. & Hageman, M. J. The expanding role of prodrugs in contemporary drug design and development. Nat Rev Drug Discov 17, 559-587, doi:10.1038/nrd.2018.46 (2018).
Kinoshita, Y. et al. Expression of prostate-specific membrane antigen in normal and malignant human tissues. World J Surg 30, 628-636, doi:10.1007/s00268-005-0544-5 (2006).
Silver, D. A., Pellicer, I., Fair, W. R., Heston, W. D. & Cordon-Cardo, C. Prostate-specific membrane antigen expression in normal and malignant human tissues. Clin Cancer Res 3, 81-85 (1997).
Wang, X., Ma, D., Olson, W. C. & Heston, W. D. In vitro and in vivo responses of advanced prostate tumors to PSMA ADC, an auristatin-conjugated antibody to prostate-specific membrane antigen. Mol Cancer Ther 10, 1728-1739, doi:10.1158/1535-7163.MCT-11-0191 (2011).
Ma, D. et al. Potent antitumor activity of an auristatin-conjugated, fully human monoclonal antibody to prostate-specific membrane antigen. Clin Cancer Res 12, 2591-2596, doi:10.1158/1078-0432.CCR-05-2107 (2006).
Bartlett, N. L. et al. Retreatment with brentuximab vedotin in patients with CD30-positive hematologic malignancies. J Hematol Oncol 7, 24, doi:10.1186/1756-8722-7-24 (2014).
Lu, J., Jiang, F., Lu, A. & Zhang, G. Linkers Having a Crucial Role in Antibody-Drug Conjugates. Int J Mol Sci 17, 561, doi:10.3390/ijms17040561 (2016).
Vigneswaran, N. et al. Variable expression of cathepsin B and D correlates with highly invasive and metastatic phenotype of oral cancer. Hum Pathol 31, 931-937, doi:10.1053/hupa.2000.9035 (2000).
Petrylak, D. P. et al. Phase 1 study of PSMA ADC, an antibody-drug conjugate targeting prostate-specific membrane antigen, in chemotherapy-refractory prostate cancer. Prostate 79, 604-613, doi:10.1002/pros.23765 (2019a).
Masters, J. C., Nickens, D. J., Xuan, D., Shazer, R. L. & Amantea, M. Clinical toxicity of antibody drug conjugates: a meta-analysis of payloads. Invest New Drugs 36, 121-135, doi:10.1007/s10637-017-0520-6 (2018).
Banerjee, S. R. et al. Sequential SPECT and optical imaging of experimental models of prostate cancer with a dual modality inhibitor of the prostate-specific membrane antigen. Angew Chem Int Ed Engl 50, 9167-9170, doi:10.1002/anie.201102872 (2011).
Dubowchik, G. M. et al. Cathepsin B-labile dipeptide linkers for lysosomal release of doxorubicin from internalizing immunoconjugates: model studies of enzymatic drug release and antigen-specific in vitro anticancer activity. Bioconjug Chem 13, 855-869 (2002).
Chen, Y. et al. Radiohalogenated prostate-specific membrane antigen (PSMA)-based ureas as imaging agents for prostate cancer. J Med Chem 51, 7933-7943, doi:10.1021/jm801055h (2008).
Mease, R. C. et al. N—[N—[(S)-1,3-Dicarboxypropyl]carbamoyl]-4-[18F]fluorobenzyl-L-cysteine, [18F]DCFBC: a new imaging probe for prostate cancer. Clin Cancer Res 14, 3036-3043, doi:10.1158/1078-0432.CCR-07-1517 (2008).
Chang, S. S. et al. Five different anti-prostate-specific membrane antigen (PSMA) antibodies confirm PSMA expression in tumor-associated neovasculature. Cancer Res 59, 3192-3198 (1999).
Dorywalska, M. et al. Molecular Basis of Valine-Citrulline-PABC Linker Instability in Site-Specific ADCs and Its Mitigation by Linker Design. Mol Cancer Ther 15, 958-970, doi:10.1158/1535-7163.MCT-15-1004 (2016).
Anami, Y. et al. Glutamic acid-valine-citrulline linkers ensure stability and efficacy of antibody-drug conjugates in mice. Nat Commun 9, 2512, doi:10.1038/s41467-018-04982-3 (2018).
Qi, R. et al. Nanoparticle conjugates of a highly potent toxin enhance safety and circumvent platinum resistance in ovarian cancer. Nat Commun 8, 2166, doi:10.1038/s41467-017-02390-7 (2017).
Donaghy, H. Effects of antibody, drug and linker on the preclinical and clinical toxicities of antibody-drug conjugates. MAbs 8, 659-671, doi:10.1080/19420862.2016.1156829 (2016).
Horoszewicz, J. S., Kawinski, E. & Murphy, G. P. Monoclonal antibodies to a new antigenic marker in epithelial prostatic cells and serum of prostatic cancer patients. Anticancer Res 7, 927-935 (1987).
Carter, R. E., Feldman, A. R. & Coyle, J. T. Prostate-specific membrane antigen is a hydrolase with substrate and pharmacologic characteristics of a neuropeptidase. Proc Natl Acad Sci USA 93, 749-753, doi:10.1073/pnas.93.2.749 (1996).
Pomper, M. G. et al. 11C-MCG: synthesis, uptake selectivity, and primate PET of a probe for glutamate carboxypeptidase II (NAALADase). Mol Imaging 1, 96-101, doi:10.1162/153535002320162750 (2002).
Foss, C. A. et al. Radiolabeled small-molecule ligands for prostate-specific membrane antigen: in vivo imaging in experimental models of prostate cancer. Clin Cancer Res 11, 4022-4028, doi:10.1158/1078-0432.CCR-04-2690 (2005).
Wone, D. W., Rowley, J. A., Garofalo, A. W. & Berkman, C. E. Optimizing phenylethylphosphonamidates for the inhibition of prostate-specific membrane antigen. Bioorg Med Chem 14, 67-76, doi:10.1016/j.bmc.2005.07.056 (2006).
Kularatne, S. A., Wang, K., Santhapuram, H. K. & Low, P. S. Prostate-specific membrane antigen targeted imaging and therapy of prostate cancer using a PSMA inhibitor as a homing ligand. Mol Pharm 6, 780-789, doi:10.1021/mp900069d (2009).
Yao, D. et al. The utility of monoclonal antibodies in the imaging of prostate cancer. Semin Urol Oncol 20, 211-218, doi:10.1053/suro.2002.36250 (2002).
Psimadas, D., Valotassiou, V., Alexiou, S., Tsougos, I. & Georgoulias, P. Radiolabeled mAbs as Molecular Imaging and/or Therapy Agents Targeting PSMA. Cancer Invest 36, 118-128, doi:10.1080/07357907.2018.1430816 (2018).
Von Hoff, D. D. et al. Phase I Study of PSMA-Targeted Docetaxel-Containing Nanoparticle BIND-014 in Patients with Advanced Solid Tumors. Clin Cancer Res 22, 3157-3163, doi:10.1158/1078-0432.CCR-15-2548 (2016).
Autio, K. A. et al. Safety and Efficacy of BIND-014, a Docetaxel Nanoparticle Targeting Prostate-Specific Membrane Antigen for Patients With Metastatic Castration-Resistant Prostate Cancer: A Phase 2 Clinical Trial. JAMA Oncol 4, 1344-1351, doi:10.1001/jamaoncol.2018.2168 (2018).
Chen, Y. et al. A PSMA-targeted theranostic agent for photodynamic therapy. J Photochem Photobiol B 167, 111-116, doi:10.1016/j.jphotobiol.2016.12.018 (2017).
Lutje, S. et al. Development and characterization of a theranostic multimodal anti-PSMA targeting agent for imaging, surgical guidance, and targeted photodynamic therapy of PSMA-expressing tumors. Theranostics 9, 2924-2938, doi:10.7150/thno.35274 (2019).
Wang, X. et al. Theranostic Agents for Photodynamic Therapy of Prostate Cancer by Targeting Prostate-Specific Membrane Antigen. Mol Cancer Ther 15, 1834-1844, doi:10.1158/1535-7163.MCT-15-0722 (2016).
Rahbar, K. et al. German Multicenter Study Investigating 177Lu-PSMA-617 Radioligand Therapy in Advanced Prostate Cancer Patients. J Nucl Med 58, 85-90, doi:10.2967/jnumed. 116.183194 (2017).
Yadav, M. P., Ballal, S., Sahoo, R. K., Dwivedi, S. N. & Bal, C. Radioligand Therapy With (177)Lu-PSMA for Metastatic Castration-Resistant Prostate Cancer: A Systematic Review and Meta-Analysis. AJR Am J Roentgenol 213, 275-285, doi:10.2214/AJR.18.20845 (2019).
Hofman, M. S. et al. [(177)Lu]-PSMA-617 radionuclide treatment in patients with metastatic castration-resistant prostate cancer (LuPSMA trial): a single-centre, single-arm, phase 2 study. Lancet Oncol 19, 825-833, doi:10.1016/S1470-2045(18)30198-0 (2018).
Aghdam, R. A. et al. Efficacy and safety of (177)Lutetium-prostate-specific membrane antigen therapy in metastatic castration-resistant prostate cancer patients: First experience in West Asia—A prospective study. World J Nucl Med 18, 258-265, doi:10.4103/wjnm.WJNM_66_18 (2019).
Fendler, W. P., Rahbar, K., Herrmann, K., Kratochwil, C. & Eiber, M. (177)Lu-PSMA Radioligand Therapy for Prostate Cancer. J Nucl Med 58, 1196-1200, doi:10.2967/jnumed.117.191023 (2017).
Kratochwil, C. et al. Targeted alpha-Therapy of Metastatic Castration-Resistant Prostate Cancer with (225)Ac-PSMA-617: Swimmer-Plot Analysis Suggests Efficacy Regarding Duration of Tumor Control. J Nucl Med 59, 795-802, doi:10.2967/jnumed.117.203539 (2018a).
Kratochwil, C. et al. Targeted alpha therapy of mCRPC: Dosimetry estimate of (213)Bismuth-PSMA-617. Eur J Nucl Med Mol Imaging 45, 31-37, doi:10.1007/s00259-017-3817-y (2018b).
Kularatne, S. A. et al. Synthesis and biological analysis of prostate-specific membrane antigen-targeted anticancer prodrugs. J Med Chem 53, 7767-7777, doi:10.1021/jm100729b (2010).
Denmeade, S. R. et al. Engineering a prostate-specific membrane antigen-activated tumor endothelial cell prodrug for cancer therapy. Sci Transl Med 4, 140ra186, doi:10.1126/scitranslmed.3003886 (2012).
Mahalingam, D. et al. A Phase II, Multicenter, Single-Arm Study of Mipsagargin (G-202) as a Second-Line Therapy Following Sorafenib for Adult Patients with Progressive Advanced Hepatocellular Carcinoma. Cancers (Basel) 11, doi:10.3390/cancers11060833 (2019).
Petrylak, D. P. et al. PSMA ADC monotherapy in patients with progressive metastatic castration-resistant prostate cancer following abiraterone and/or enzalutamide: Efficacy and safety in open-label single-arm phase 2 study. Prostate, doi:10.1002/pros.23922 (2019b).
Kiess, A. P. et al. Auger Radiopharmaceutical Therapy Targeting Prostate-Specific Membrane Antigen. J Nucl Med 56, 1401-1407, doi:10.2967/jnumed.115.155929 (2015).
Ovacik, M. & Lin, K. Tutorial on Monoclonal Antibody Pharmacokinetics and Its Considerations in Early Development. Clin Transl Sci 11, 540-552, doi:10.1111/cts.12567 (2018).
Chu, D. S., Johnson, R. N. & Pun, S. H. Cathepsin B-sensitive polymers for compartment-specific degradation and nucleic acid release. J Control Release 157, 445-454, doi:10.1016/j.jconrel.2011.10.016 (2012).

Although the foregoing subject matter has been described in some detail by way of illustration and example for purposes of clarity of understanding, it will be understood by those skilled in the art that certain changes and modifications can be practiced within the scope of the appended claims.

1. A compound comprising a PSMA-targeting moiety (T), a cleavable linker (L₁), and an antineoplastic agent (A) of formula (I):
A-L₁-T (I).

2. The compound of claim 1, further comprising a non-cleavable linker (L₂), wherein the compound of formula (I) has the following general structure:
A-L₁-L₂-T (I).

3. The compound of claim 1, further comprising a spacer (S), wherein the compound of formula (I) has the following general structure:
A-S-L₁-T (I).

4. The compound of claim 1, further comprising a non-cleavable linker (L₂) and a spacer (S), wherein the compound of formula (I) has the following general structure:
A-S-L₁-L₂-T (I).

5. The compound of any one of claim 1, wherein the PSMA-targeting moiety comprises a lysine (Lys)-Urea-glutamate (Glu)-based PSMA targeting moiety.

6. The compound of claim 5, wherein the lysine (Lys)-Urea-glutamate (Glu)-based PSMA targeting moiety comprises: 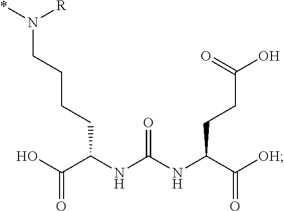
wherein * denotes a point of attachment to the non-cleavable linker (L₂) or the cleavable linker (L₁) and wherein R is H or —CH₂—R₁, wherein R₁ is selected from the group consisting of aryl substituted with one or more halogen, pyridine substituted with one or more halogen, and isoquinoline.

7. The compound of claim 6, wherein R₁ is selected from the group consisting of: 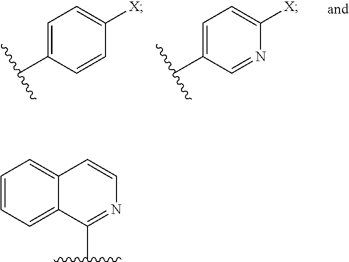
wherein each X is independently Br or I.

8. The compound of claim 2, wherein the non-cleavable linker is derived from disuccinimidyl suberate or polyethylene glycol (PEG).

9. The compound of claim 8, wherein the non-cleavable linker is selected from the group consisting of: —(CH₂)ₙ and —(O—CH₂CH₂)ₘO—, wherein m and n are each independently an integer selected from the group consisting of 0, 1, 2, 3, 4, 5, 6, 7, 8, 9, 10, 11 and 12.

10. The compound of claim 2, wherein a combination of the PSMA-targeting moiety (T) and the non-cleavable linker (L₂) comprises: 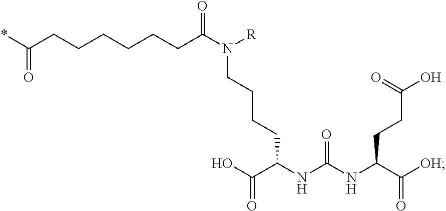
wherein * denotes a point of attachment to the cleavable linker (L₁).

11. The compound of claim 1, wherein the cleavable linker (L₁) comprises a cathepsin-cleavable linker.

12. The compound of claim 11, wherein the cathepsin-cleavable linker comprises one or more amino acids selected from the group consisting of valine (Val), citrulline (Cit), phenylalanine (Phe), lysine (Lys), glycine (Gly), alanine (Ala), asparagine (Asn), and combinations thereof.

13. The compound of claim 12, wherein the cathepsin-cleavable linker comprises an amino acid combination selected from the group consisting of Val-Cit, Phe-Lys, Val-Ala, Val-Gly, Gly-Gly, Gly-Gly-Gly, and Ala-Ala-Asn.

14. The compound of claim 13, wherein the cathepsin-cleavable linker comprises Val-Cit and has the following structure: 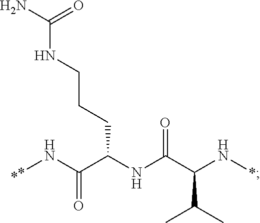
wherein * denotes a point of attachment to the PSMA-targeting moiety or a combination of the PSMA-targeting moiety (T) and the non-cleavable linker (L₂) and ** denotes a point of attachment to the antineoplastic agent (A).

15. The compound of claim 3, wherein the spacer (S) comprises a moiety selected from the group consisting of (para-aminobenzylcarbamate) (PABC), a precursor derived from para-nitrophenol (PNP), and combinations thereof.

16. The compound of claim 4, wherein a combination of the cleavable linker (L₁) and the spacer (S) comprises: 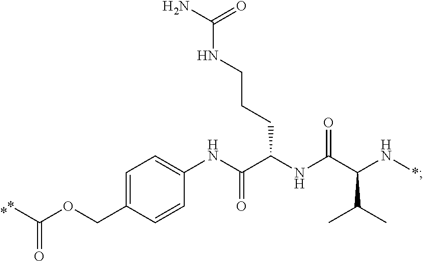
wherein * denotes a point of attachment to the PSMA-targeting moiety or a combination of the PSMA-targeting moiety (T) and the non-cleavable linker (L₂) and ** denotes a point of attachment to the antineoplastic agent (A).

17. The compound of claim 4, wherein a combination of the cleavable linker (L₁) and the spacer (S) comprises: 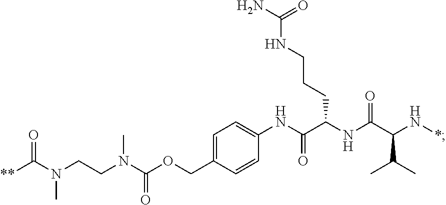
wherein * denotes a point of attachment to the PSMA-targeting moiety or a combination of the PSMA-targeting moiety (T) and the non-cleavable linker (L₂) and ** denotes a point of attachment to the antineoplastic agent (A).

18. The compound of claim 1, wherein the antineoplastic agent comprises a cytotoxic agent.

19. The compound of claim 18, wherein the cytotoxic agent comprises a naturally-occurring or a synthetic tubulin inhibitor.

20. The compound of claim 19, wherein the cytotoxic agent is selected from the group consisting of monomethyl auristatin E (MMAE), monomethyl auristatin F (MMAF), monomethyl Dolastatin 10, maytansinoid, DM1, DM4, cabazitaxel, paclitaxel, and 7-ethyl-10-hydroxycamptothecin (SN-38), one or more topoisomerase inhibitors, plant derived natural phenols, one or more PARP inhibitors, one or more amatoxins, and Pseudomonas exotoxin A.

21. The compound of claim 19, wherein the synthetic tubulin inhibitor comprises monomethyl auristatin E (MMAE).

22. The compound of claim 1, wherein the compound of formula (I) comprises:

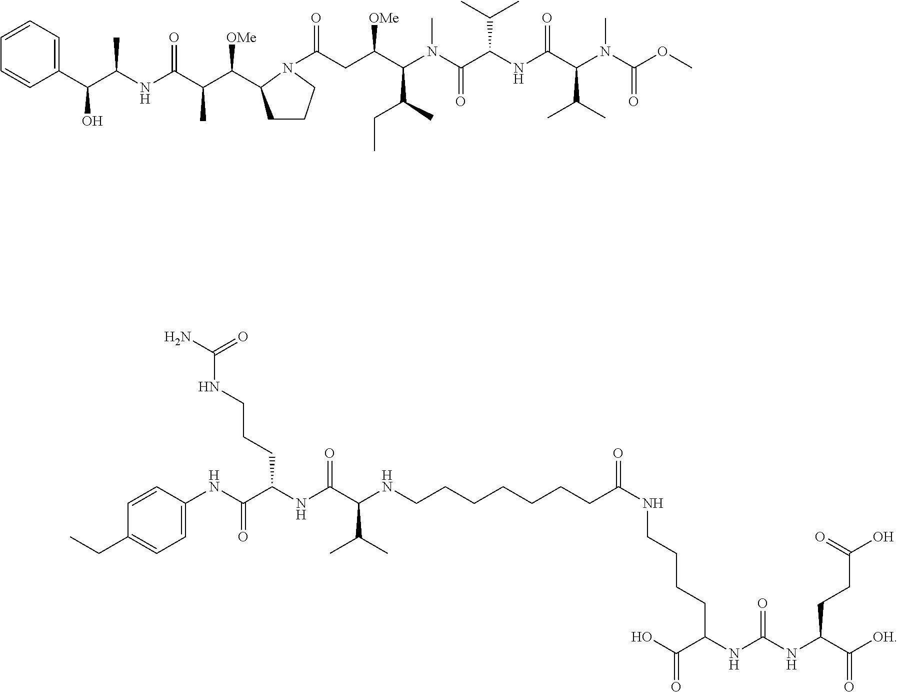

23. A method for treating a disease or condition associated with one or more PSMA expressing tumors or cells, the method comprising administering a therapeutically effective amount of a compound of claim 1, to a subject in need of treatment thereof.

24. The method of claim 23, wherein the disease or condition comprises a cancer.

25. The method of claim 24, wherein the cancer is selected from the group consisting of prostate cancer, renal cancer, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, adenomas, and tumor neovasculature.

26. The method of claim 25, wherein the cancer comprises prostate cancer.

27. The method of claim 26, wherein the prostate cancer comprises metastatic castration-resistant prostate cancer.

28. The method of claim 25, wherein the cancer comprises breast cancer.

29. The method of claim 23, further comprising administering a compound of formula (I) in combination with one or more additional cancer treatments.

30. The method of claim 29, wherein the one or more additional cancer treatments is selected from the group consisting of chemotherapy, radiotherapy, immunotherapy, proton therapy, photodynamic therapy, and surgery.

31. The method of claim 23, further comprising administering a compound of formula (I) as its pharmaceutically acceptable salt.
